(19) United States
(12) Patent Application Publication
(10) Pub. No.: US 2026/0263514 A1
(43) Pub. Date: Sep. 10, 2026

(54) DISRUPTION OF TELOCYTE ACTIVITY

(71) Applicant: Amit PATEL, Miami, FL (US)

(72) Inventor: Amit PATEL, Miami, FL (US)

(21) Appl. No.: 18/876,455

(22) PCT Filed: Jul. 31, 2023
(86) PCT No.: PCT/US2023/071337

§ 371 Date: Dec. 18, 2024

Related U.S. Application Data
(60) Provisional application No. 63/393,604, filed on Jul. 29, 2022.

(51) Int. Cl. A61K 35/17; A61K 35/15; A61K 39/00; A61K 40/11; A61K 40/31; A61K 40/42; C07K 14/705; C07K 16/28; C12N 5/0783; C12N 5/0784

(52) U.S. Cl. CPC A61K 35/17; A61K 35/15; A61K 39/0005; A61K 40/11; A61K 40/31; A61K 40/4224; C07K 14/7051; C07K 14/70517; C07K 14/70578; C07K 16/2896; C12N 5/0636; C12N 5/0639; A61K 2039/5154; A61K 2239/13; A61K 2239/17; A61K 2239/21; A61K 2239/29; A61K 2239/55; C07K 2317/31; C07K 2317/53; C07K 2317/622; C07K 2319/03

(57) ABSTRACT

The present disclosure is drawn to compositions, combinations of compounds, and methods of treating a cancer or neoplasia, including disrupting activity of telocytes associated with the cancer.

### BACKGROUND

Cancer therapy has historically been associated with many negative side-effects associated with chemotherapy and/or radiation. Newer approaches have been focused on activities of cancer which are more targeted so that the treatment of cancerous tissue ideally would not significantly impact normal tissue of the subject being treated. While chemotherapy and radiation therapy can target more rapidly proliferating cells in many instances, other therapeutic approaches, such as angiogenesis inhibition (or inhibitions of the development of new blood vessels), offer a significantly higher degree of selectively in terms of reduced adverse effects, particularly in adults, as adults do not rely as heavily on angiogenic as they do proliferating cells (cellular growth and division).

### DETAILED DESCRIPTION

The foundational building material of the body includes cells, tissues, and organs. Cells of a specific type grouped together form tissue, and various types of tissue organized together to perform complex functions are referred to as organs. Thus, within tissues, there are cells that carry out biological functions that can support the complex functions unique to a given organ. On the other hand, there are also cells that are present in tissue and organs that provide structural or connection between cells that make up tissues and organs. Structural or connective cells are referred to as Stroma cells. One major type of stroma cells is fibroblasts, which generate connective tissue matrix, e.g., collagen. A second type of stroma cells is referred to as telocytes. Telocytes are not as involved in the physical framework or connection of tissues and organs, but rather are positioned between other types of cells to provide intracellular signaling. More specifically, telocytes are identified among interstitial cells and include very long cell processes up to hundreds of microns in length referred to as telopodes. The elongated nature of the telopodes provide close proximity to multiple tissue and/or organ cells, allowing for efficient signaling between the tissue and/or organ cells.

As telocytes are involved in signaling between cells of tissues/organs, the present disclosure relates to the leveraging of the telocyte signaling for the treatment of neoplasia (or abnormal growth of tissue that may or may not be cancerous) by disrupting certain telocytes at or about tumors or tumor environments and/or leveraging telocytes or telocyte activity for use in vaccines that can be used to target tumors, for example. By disrupting the normal signaling of telocytes, neoplasia may be arrested, reduced, regressed, and/or reversed. Disrupting telocyte activity (or function) may be by reducing, inactivating, or reversing the telocyte activity, or even killing (causing cellular death) of the telocytes that are targeted in some instances. Lack of signaling can have an adverse impact on cancerous cells in a tumor, and thus can be used to treat tumors. Furthermore, telocytes can be used to generate an immune response against neoplasia, including cancerous cells and tumors, as also described in greater detail below.

Thus, the present disclosure relates to the leveraging of the telocyte signaling for the treatment of neoplasia (or abnormal growth of tissue that may or may not be cancerous) by disrupting certain telocytes at or about tumors or tumor environments. By disrupting the normal signaling of telocytes, neoplasia may be arrested, reduced, regressed, and/or reversed. Disrupting telocyte activity (or function) may be by reducing, inactivating, or reversing the telocyte activity, or even killing (causing cellular death) of the telocytes that are targeted in some instances.

In accordance with this and other disclosures herein, various compositions, combinations of compounds, and methods of treating a neoplasia, e.g., cancer, including disrupting activity of telocytes associated with the neoplasia are provided. More specifically, treating cancer can include disrupting activity of telocytes associated with the cancer. Disrupting the activity of the telocytes can be in the form of telocyte cellular death, or by inhibition or inactivation of telocyte signal pathways

In another example, transfected telocyte lysate can include a lysate of telocytes transfected with one or more of interleukin-1 β, interleukin-2, interleukin-4 and GM-CSF, GM-CSF, complement component c3, complement component c5, interleukin-7, interleukin-11, interleukin-12, interleukin-18, interleukin-21, interleukin-23, interleukin-27, interleukin-33, interferon α, interferon γ, interferon tau, interferon omega, TRAIL, BLyS, LIGHT, TNF-α, lymphotoxin, influenza surface antigen, PADRE epitope, HMGB1, short interfering RNA to interleukin-10, short interfering RNA to TGF-β, short interfering RNA to HLA-G, short interfering RNA to ILT-3, short interfering RNA to ILT-4, short interfering RNA to interleukin-1 receptor antagonize, short interfering RNA to interleukin-35, short interfering RNA to placental growth factor, short interfering RNA to VEGF, short interfering RNA to PDGF, short interfering RNA to sonic hedgehog, short interfering RNA to notch, and/or short interfering RNA to jagged, for example. In other examples, the telocyte population can be gene edited to lack express of interleukin-10, TGF-β, HLA-G, ILT-3, ILT-4, interleukin-1 receptor antagonist, interleukin-35, placental growth factor, VEGF, PDGF, sonic hedgehog, notch, or jagged. These transfected telocyte lysates can be formulated in a cancer therapeutic vaccine. As an example, the telocytes may be transfected with at least GM-CSF, in one example. In a more specific example, the telocytes can be transfected with at least GM-CSF and the transfected telocyte lysate may be formulated in a cancer therapeutic vaccine.

In another example, a hybrid cell composition can include a leukocyte-containing cellular culture including monocytes, dendritic cells, effector T cells, or a combination thereof, and can further include a telocyte lysate fused with cells or cellular material of the leukocyte-containing cellular culture. In some examples, these hybrid cell compositions can express CD34 along with vimentin, vascular endothelial growth factor (VEGF), or both. In some examples, the leukocyte-containing cellular culture includes at least 50% dendritic cells by cell count, at least 50% monocytes by cell count, or at least 50% effector T cells by cell count. In other examples, dendritic cells may be present as immature dendritic cells. In other examples, the telocyte lysate can be derived from telocytes from cancer tissue. Other compounds may be present in the hybrid cell composition, such as any of a number of therapeutic compounds or other components, such as compounds capable of reducing tumor-associated immune suppression and/or inducing localized tumor death. In other examples, a cancer growth media capable of allowing for growth of cancer specific telocytes can be used, such as DMEM media, AIM-V media, RPMI media, EMEM media, Iscove's media, or a combination thereof. In some examples, a stimulator of antigen presentation can be included that is capable of upregulating expression of costimulatory molecules on antigen presenting cells. The hybrid cell compositions may be, for example, in the form of a vaccine, such as a GM-CSF transfected telocyte formulated as a cancer therapeutic vaccine. In other examples, the telocyte lysates can be generated from living telocytes, mitotically inactivated telocytes, telocyte necrotic particles, telocytes after pyroptosis, telocyte apoptotic bodies, and telocytes derived from pluripotent stem cells. The telocyte lysate fused with cells or cellular material of the leukocyte-containing cellular culture can be prepared by: electrically pulse-fusing the telocyte lysate with the cells or cellular material of the leukocyte-containing cellular culture, ultrasonically pulse-fusing the telocyte lysate with the cells or cellular material of the leukocyte-containing cellular culture, and/or treating the telocyte lysate and the cells or cellular material of the leukocyte-containing cellular culture with polyethylene glycol, for example. The telocyte lysate of any of these compositions may be sourced from telocytes grown in vitro. In some examples, the telocytes may be cultured in the presence of one or more of TGF-β, PGE2, sialic acid, soluble TNF-α receptor p55, soluble TNF-α receptor p75, siglec-15, Galectin-1 Galectin-3, Galectin-7, Galectin-9, FGF-1, FGF-2, FGF-5, FGF-18, IGF-1 EGF-1, HGF, IL-17, IL-10, IL-4, IL-13, IL-35, VEGF-A, VEGF-C, PDGF-BB, angiopoietin, neutrophil extracellular traps, growth hormone, soluble HLA-G, carbon monoxide, radon gas, cobalt chloride, GITR ligand, RAE-1, embryonic stem cell conditioned media, GDF-5, GDF-11, thyroid stimulating hormone, oncostatin, plasminogen, and/or fibrinogen, for example. In other examples, the telocyte lysate grown in vitro may be expanded for use. Expansion can be carried out: for use as an immunogen or immunogenic source, under conditions promoting acidosis; under conditions resembling the tumor microenvironment; or a combination thereof.

In another example, a modified T cell can include T cells modified with chimeric multi-antigen receptors. For example, the chimeric multi-antigen receptors may be at least bi-specific for CD34/Vimentin, CD34/VEGF, and/or CD34/PDTF.

In further detail with respect to these compositions and methods described herein, telocytes can be capable of differentiating into cells expressing a smooth muscle actin. In other examples, the telocytes can express CD34, extracellular vimentin, c-kit, angiopoietin receptors, PDGFR-α, PDGFR-β, SCA-1, podoplanin, caveolin-1, one or more telopods, CAPN2, FHL2, SOX1, combinations thereof, and the like. Disrupting activity can be in the form of cellular death, inhibition of signal pathways, inhibition of receptors, etc. Examples of inhibition may include inhibition with one of a number of MEK inhibitors. In some examples, ERK signaling can be inhibited, such as by: induction of RNA interference against the ERK mRNA, e.g., by treatment with short interfering RNA or short hairpin RNA; administration of antisense oligonucleotides targeting ERK mRNA; administration of ribozymes targeting ERK mRNA; induction of gene editing targeting the ERK gene; and administration of a small molecule ERK inhibitor (including ER3K inhibitors). The ERK inhibitor can include, for example, PD0325901, RDEA119, Olomoucine, Aminopurvalanol A, AS703026, AZD8330, BIX02188, BIX02189, CI-1040, Cobimetirlib, GDCs-0623, MEK162, PD318088, PD98059, Refametinib, R04987655, SCH772984, Selumetinib, SL327, Trametinib, ARRY-142886, or XL518.

In some examples, the telocytes targeted (or used) may include FOXL1+, AQP1+, and/or LGR5+, for example. In some examples, the telocytes may also be such that they are positive for the estrogen receptor and/or positive for the progesterone A receptor, for example.

In further detail, the telocytes can be disrupted, e.g., inhibited, inactivated and/or killed, by contributing to the generation of an immune response. The immune response may be towards extracellular vimentin. The immune response can be induced, for example, by vaccination, e.g., administration of vimentin protein in an immunogenic manner, and administration through loading of dendritic cells in vitro followed by subsequent administration of the dendritic cells in vivo. The dendritic cells can be generated from monocytes, such as plastic adherent monocytes. The monocytes may express CD14, CD16, TLR4, TNF-α upon stimulation, CD90, c-kit, c-met, CD25, PDGF-receptor, CD11 b, and/or BDNF-receptor, for example. In some examples, the monocytes can be treated with IL-4 and GM-CSF ex vivo to generate immature dendritic cells. The immature dendritic cells can express IL-10, CD11c, lower levels of CD40 as compared to mature dendritic cells, lower levels of CD80 as compared to mature dendritic cells, lower levels of CD86 as compared to mature dendritic cells, lower levels of IL-12 as compared to mature dendritic cells, lower levels of IL-21 as compared to mature dendritic cells, lower levels of IL-1S as compared to mature dendritic cells, lower levels of IL-33 as compared to mature dendritic cells, lower levels of IL-15 as compared to mature dendritic cells, higher levels of IL-35 as compared to mature dendritic cells, higher levels of TGF-β as compared to mature dendritic cells, higher levels of HLA-G as compared to mature dendritic cells, lower levels of AIM2 as compared to mature dendritic cells, higher levels of ILT-3 as compared to mature dendritic cells, higher levels of ILT-4 as compared to mature dendritic cells, higher levels of LIF as compared to mature dendritic cells, higher levels of inhibitor of kappa B as compared to mature dendritic cells, higher levels of soluble TNF-α receptor as compared to mature dendritic cells, and/or higher levels of interleukin-1 receptor antagonist as compared to mature dendritic cells, for example.

In some examples, the dendritic cells can be induced to mature after administration in vivo, such as by administration of Poly (IC), imiquimod, HMGB-1, CpG motifs, xenogeneic cell membranes, bacterial cell wall extract, β-glucan, OK231, GM-CSF, neutrophil extracellular traps, free histones, yeast cell wall extract, KLH, zymosan, interferon γ, antibodies to IL-10 or its receptor, TNF-α, IL-33, β-defensin, complement C3, complement C5, and/or necrotic cells, for example.

In further detail, the disruption of telocyte activity, e.g., telocyte inhibition, inactivation, or cellular death, can be accomplished with tumor telocytes, such as tumor telocytes that possess an increased ability to efflux rhodamine 123 as compared to telocytes isolated from non-malignant tissues. This may be in the form of a vaccine, which may be, for example, prophylactic, therapeutic, autologous, allogeneic, or xenogeneic. The vaccine can be generated from living telocytes, mitotically inactivated telocytes, telocyte necrotic particles, telocytes that have undergone the process of pyroptosis, telocyte apoptotic bodies, fusion of telocytes with cells associated with immunity, such as monocytes, dendritic cells, effector T cells (including cytotoxic T cells), or a combination thereof. In one example, the lysed telocytes may be fused with dendritic cells.

“Monocytes” are a type of white blood cell that is formed in the bone marrow and helps fight infection and inflammation, and are part of the innate immune response. They can differentiate macrophages (cells that engulf and destroy foreign substances) and protect the body from various microbes or germs.

“Dendritic cells” (DCs) are antigen-presenting cells of the immune system that process antigen material and present it on the cell of a T cell of the immune system. Dendritic cells are used for primary T cell activation, for example. They can sample a peripheral microenvironment and migrate to activate naïve lymphocytes.

“Effector T cells” are groups of cells that include multiple types of T cells that actively respond to stimulus, such as co-stimulation, and include regulatory T cells (to stop T cell-mediated immune response at the end of an immune reaction), helper T cells (help other leukocytes in immunologic processes, including the maturation of B cells in plasma and memory B cells), and cytotoxic T cells (or killer T cells, which destroy virus-infected cells and tumor cells), for example.

As mentioned, telocytes can be fused with cells associated with immunity, such as monocytes, dendritic cells, effector T cells, etc. However, many of the embodiments herein are described with the fusion of telocytes with dendritic cells for simplicity. It is understood that many of the exemplified embodiments and descriptions here may substitute or supplement the use of dendritic cells with other types of cells associated with immunity, such as many of the different types of white blood cells, such as monocytes and/or lymphocytes, e.g., any of a number of T cells, effector T cells, etc. Thus, examples here that specifically recite fusion of telocytes with dendritic cells can be modified to include any of a number of monocytes and/or lymphocytes.

With respect to the fusion of telocytes with dendritic cells (or monocytes, effector T cells, etc.) to form hybrid cells or hybridomas, this fusion can be accomplished by electroporation, e.g., electrical pulsing, sonoporation, e.g., ultrasonic pulsing, treatment with polyethylene glycol using telocytes derived from pluripotent stem cells, e.g., inducible pluripotent stem cells, embryonic stem cells, somatic nuclear transfer derived stem cells, and/or parthenogenic derived stem cells. Alternatively, the telocyte fusion with the dendritic cells (or monocytes, effector T cells, etc.) can be accomplished using telocytes derived from hematopoietic stem cells that express, for example, CD34, CD133, Fas ligand, TRAIL receptor, AIM2 and/or notch. In other examples, the fusion can be performed using dendritic cells (or others) derived from pluripotent stem cells, such as those that are inducible pluripotent stem cells, embryonic stem cells, somatic nuclear transfer derived stem cells, and/or parthenogenic derived stem cells. In still other examples, the fusion of telocytes with dendritic cells (or others) can be performed using cells derived from hematopoietic stem cells that express, for example, CD34, CD133, Fas ligand, TRAIL receptor, AIM2, and/or notch.

Hybrid cells (hybridomas) from fusion of telocytes and dendritic cells (or monocytes, effector T cells, etc.) can be activated prior to administration in a manner capable of increasing immunogenicity, such as the ability to evoke recall T cell immune response to one or more cancer telocyte induced antigens, recall CD4 T cell immune response to one or more cancer telocyte induced antigens, recall CD8 T cell immune response to one or more cancer telocyte induced antigens, recall NK cell immune response to one or more cancer telocyte induced antigens, recall NKT cell immune response to one or more cancer telocyte induced antigens, recall γ delta cell immune response to one or more cancer telocyte induced antigens, and/or recall neutrophil immune response to one or more cancer telocyte induced antigens. The hybrid cells can be increased by exposure to a compound that increases transporter associated protein expression, such as interferon α, interferon γ, TNF-α, interleukin-6, interleukin-12, and/or interleukin-18, for example. Immunogenicity of the hybrid cells can be increased by exposure to compounds or agents that increase MHC I and/or MHC II expression, such as interferon α, interferon γ, TNF-α, interleukin6, interleukin-12, and/or interleukin-18. In other examples, immunogenicity of the hybrid cells can be increased by exposure to compounds or agents that increase exposure to one or more compounds capable of triggering: the cGAS-STING pathway, the NOD pathway, and/or the toll-like receptor (TLR) pathway, for example.

In some examples, the telocyte is grown in vitro and expanded before use as an immunogen or immunogenic source. The telocyte may be isolated from one or more primary tumor sources, and/or may be isolated and mechanically separated into smaller pieces than the original size of the tumor mass. For example, the tumor tissue may be separated and mechanically dissected into multiple 1-3 mm pieces, e.g., 2-5 pieces. The tumor may be treated with a tissue dissociating compound, such as collagenase, trypsin/TrypLE, a combination of collagenase and trypsin/TrypLE, e.g., 0.05% collagenase and 0.1% trypsin/TrypLE (v/v). The tissue may be allowed to digest with incubation to allow enzymes to digest the tissue, e.g., for 2-10 minutes. Enzyme incubation may be in the presence of agitation, e.g., a shaker. Further dissociation can occur by gentle pipetting, e.g., from 1-30 minute intervals. Supernatant can be collected and/or filtered with mesh(es), e.g., from 20-200 μm mesh such as sequentially using 100 μm and 41 μm nylon mesh. Collected cell suspension can be centrifuged at from 10-150×g for 30 seconds to 5 minutes, for example. Supernatant can be separated and centrifuged (or re-centrifuged) at from 100-700×g for 20 seconds to 17 minutes. The pellet(s) obtained after centrifugation can be re-suspended in 1-15 mL of PEB medium, e.g., PBS supplemented with 0.01 to 1% (v/v) bovine serum albumin and 1-4 mM EDTA, which may have a relatively neutral pH of about 7.2, for example). The mixture of PEB and the re-suspended pellet can be subsequently centrifuged at 20-45×g for 1-5 minutes and then supernatant subsequently centrifuged at 100-300×g for 5-20 minutes. The cell pellet can likewise be subsequently admixed with from 0.01 mL to 10 mL of PEB and 1-5-μL of antibody to c-kit, and in some examples, incubated at 1-10° C. for 10-200 minutes. In some examples, an additional 1-10 mL of PEB is added, which can be centrifuged at 100-700×g for 1-13 minutes. The pellet that results can be collected, and then in some instances, re-suspended in 10-1000 μL of PEB and 1-200 μL of a solution containing goat anti-rabbit IgG microbeads. This particular re-suspension mixture can be incubated at 1-10° C. for 25-30 minutes. This or other mixtures can be added to a magnetic separation (MS) column in a magnetic field, and unlabeled cells can be allowed to pass through. The MS column can then be removed from the magnetic field and the labeled cells flushed out with PEB. The cells may be washed and cultured in a media capable of allowing for growth of cancer specific telocytes.

Media capable of allowing for growth of cancer specific telocytes includes DMEM containing 20% fetal calf serum, IM-V media containing 20% fetal calf serum, RPMI media containing 20% fetal calf serum, EMEM media containing 20% fetal calf serum, and/or Iscove's media containing 20% fetal calf serum, human platelet lysate is also replaced for fetal calf serum in equal amounts, for example. The cancer specific telocytes can be utilized as a cellular vaccine in an either therapeutic or prophylactic manner. The cellular vaccine can include or consist essentially of telocyte population that has been gene transfected to augment immunogenicity. This telocyte population can be mitotically inactivated, which can be accomplished by exposure to ionizing radiation and/or exposure to a chemotherapeutic compound, e.g., mitomycin-C. In other examples, the telocyte population can be gene transfected with interleukin-1 β, interleukin-2, interleukin-4 and GM-CSF, GM-CSF, complement component c3, complement component c5, interleukin-7, interleukin-11, interleukin-12, interleukin-18, interleukin-21, interleukin-23, interleukin-27, interleukin-33, interferon α, interferon γ, interferon tau, interferon omega, TRAIL, BLyS, LIGHT, TNF-α, lymphotoxin, influenza surface antigen, PADRE epitope, HMGB1, short interfering RNA to interleukin-10, short interfering RNA to TGF-β, short interfering RNA to HLA-G, short interfering RNA to ILT-3, short interfering RNA to ILT-4, short interfering RNA to interleukin-1 receptor antagonize, short interfering RNA to interleukin-35, short interfering RNA to placental growth factor, short interfering RNA to VEGF, short interfering RNA to PDGF, short interfering RNA to sonic hedgehog, short interfering RNA to notch, and/or short interfering RNA to jagged, for example. In other examples, the telocyte population can be gene edited to lack express of interleukin-10, TGF-β, HLA-G, ILT-3, ILT-4, interleukin-1 receptor antagonist, interleukin-35, placental growth factor, VEGF, PDGF, sonic hedgehog, notch, and/or jagged, for example.

In some examples, disrupting activity of the telocytes can be achieved through creation of a lysate based cancer telocyte vaccine, such as a vaccine generated from cancer tissue, which may be expanded under conditions resembling the tumor microenvironment. The tumor microenvironment can be replicated by culture of the tumor derived telocytes in hypoxia conditions and/or conditions promoting acidosis. Hypoxia results under conditions in the presence of oxygen at from 0.1%-10%, 0.1%-5%, 0.1%-2.5%, 0.1%-2%, 0.1%-1%, 0.5%-10%, 0.5%-7.5%, 0.5%-5%, 0.5%-2.5%, 0.5%-2%, %5%-1%, 1%-10%, 1%-7.5%, 1%-5%, 1%-2.5%, 1%-2%, 2%-10%, 2%-7.5%, 2%-5%, 2%-2.5%, 5%-10%, 5%-7.5%, 5%-6%, or 7.5%-10% (v/v) content in the incubator. Hypoxia can occur based on an exposure time from 30 minutes (min)-3 days, 30 min-2 days, 30 min-1 day, 30 min-12 hours (hrs), 30 min-8 hrs, 30 min-6 hrs, 30 min-4 hrs, 30 min-2 hrs, 30 min-1 hr, 1 hr-3 days, 1 hr-2 days, 1 hr-1 day, 1-12 hrs, 1-8 hrs, 1-6 hrs, 1-4 hrs, 1-2 hrs, 2 hrs-3 days, 2 hrs-2 days, 2 hrs-1 day, 2 hrs-12 hrs, 2-10 hrs, 2-8 hrs, 2-6 hrs, 2-4 hrs, 2-3 hrs, 6 hrs-3 days, 6 hrs-2 days, 6 hrs-1 day, 6-12 hrs, 6-8 hrs, 8 hrs-3 days, 8 hrs-2 days, 8 hrs-1 day, 8-16 hrs, 8-12 hrs, 8-10 hrs, 12 hrs-3, days, 12 hrs-2 days, 12 hrs-1 day, 12-18 hrs, 12-14 hrs, 1-3 days, or 1-2 days.

On the other hand acidosis can occur under culture conditions with a pH of less than about 7.2. Acidosis conditions can include culturing the telocytes in the presence of TGF-β, PGE2, sialic acid, soluble TNF-α receptor p55, soluble TNF-α receptor p75, siglec-15, Galectin-1, Galectin-3, Galectin-7, Galectin-9, FGF-1, FGF-2, FGF-5, FGF-18, IGF-1, EGF-1, HGF, IL-17, IL-10, IL-4, IL-13, IL-35, VEGF-A, VEGF-C, PDGF-BB, angiopoietin, neutrophil extracellular traps, growth hormone, soluble HLA-G, carbon monoxide, radon gas, cobalt chloride, GITR ligand, RAE-1, embryonic stem cell conditioned media, GDF-5, GDF-11, thyroid stimulating hormone, concostatin, plasminogen, and/or fibrinogen, for example.

In further detail, the telocytes may be utilized as a source of immunogen by extracting heat shock proteins and their bound immunogens from the telocyte.

In other examples, telocyte and/or telocyte derived immunogens can be administered into a patient with cancer in a manner to induce an immune response against cancer derived telocytes and/or immunogens derived from the telocytes. The cancer patient can be treated, for example, by: a) identifying a patient suffering from cancer who may be possessing some degree of immune suppression; b) administering a therapeutic capable of reducing tumor-associated immune suppression; c) immunizing the patient with an immunogenic composition capable of expanding immune cells with tumor-targeting ability; d) administering a localized therapeutic capable of augmenting antigen presentation of cancer associated antigens from the cancer; and e) administering a therapeutic capable of inducing at least partial localized tumor cell death. In some examples, the method can further include f) repeatedly administering an immunogenic composition capable of further expanding immune cells with tumor-targeting ability.

In accordance with this method, the therapeutic capable of reducing tumor-associated immune suppression can include an antioxidant, such as an antioxidant from beta carotene, Vitamin E, anthocyanins, selenium, catechins, lutein, and/or lycopene, for example. In some examples, the antioxidant can include n-acetylcysteine, ascorbic acid, glutathione, vitamin k3, resveratrol, α lipoic acid, quercetin, kaempferol, myricetin, apigenin, luteolin, curcumin, and/or caffeic acid, for example. In in some examples, the therapeutic can be for tumor-associated immune suppression, and may include a phosphodiesterase (PDE-5) inhibitor. The PDE-5 inhibitor can include Acetildenafi, Aildenafil, Avanafil, Benzamidenafil, Homosildenafil, Icariin, Lodenafil, Mirodenafil, Nitrosoprodenafil, Sildenafil, Sulfoaildenafil, Tadalafil, Udenafil, Vardenafil, and/or Zaprinast, for example. In other examples, the therapeutic capable of reducing tumor-associated immune suppression can be nitroglycerin and/or a compound capable of reducing VEGF. Such compounds capable of reducing VEGF include Avastin, Ciclopirox, penicillamine, tetrathiomolybdate, fish oil, selenium, green tea polyphenols, glycine, zinc, cirsimaritin, Eupafolin, Andrographolide, Procyanidin B2, Procyanidin B3, 6-O-angeloylenolin, Cyperenoic acid, Penduliflaworosin, Tylophorine, Ellagic acid, brucine, Punarnavine, Raddeanin A, Platycodin D, withanone, 4-Hydroxyphenylacetic acid, trans-ethyl p-methoxycinnamate, Decursin, decursinol angelate, and/or Artesunate, for example.

The therapeutic can be capable of reducing tumor-associated immune suppression and can alternatively or additionally include a checkpoint inhibitor, which can be a compound capable of blocking certain molecules. The molecules that the checkpoint inhibitor may block include, for example, PD-1, PD-L1, CTLA-4, LAG-3, TIGIT, KIR, indolamine 2,3 deoxygenase, NR2F6, TIM-3, ILT-3, and/or GITR.

In some examples, when a patient or subject is immunized with a tumor antigen, the tumor antigen can possess similarity to the tumor that is afflicting the patient. In other examples regarding immunization, a patient or subject can be immunized with a peptide(s) derived from tumor antigen. The peptide(s) used for immunization can be matched with HLA haplotype of the patient in need of therapy, or may be altered peptide ligands.

Regarding tumor antigen, in some examples, the tumor antigen can be derived by: a histologically similar tumor to which the patient is afflicted, lysis of histologically similar tumors, mRNA extraction of histologically similar tumors, and/or exosome extraction of histologically similar tumors. The tumor antigen may include a tumor associated protein, such as Fos-related antigen 1, LCK, FAP, VEGFR2, NA17, PDGFR-β, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, and/or Chondroitin sulfate proteoglycan 4 (CSPG4)329, for example. In some examples, immunization with the tumor antigen can be performed together with an adjuvant, such as an adjuvant that is also a stimulator antigen presentation. Examples include the use of a toll-like receptor (TLR). This TLR may include TLR-2, for example, which may be activated by Pam3cys4, Heat Killed Listeria monocytogenes (HKLM), and/or FSL-1, for example. Other TLRs may be used as well, such as TLR-3, which may be activated by Poly IC and/or double stranded RNA, which may be mammalian in origin. Examples of such double stranded RNA include that of prokaryotic origin and/or that derived from leukocyte extract, e.g. a heterogeneous composition derived from freeze-thawing of leukocytes followed by dialysis for compounds less than 15 kDa. TLR-4 can also be used, which may be activated by lipopolysaccharide, HMGB-1, a peptide derived from HMGB-1, and/or peptide possessing at least 80 percent homology to the sequence EFDVILKAAGANKVAVIKAVRGATGLGLKEAKDLVESAPAALKEGVSKDDAEALKKA LEEAGAEVEVK. Regarding HMGB-1, the peptide may include hp91, for example. The TLR may likewise include TLR-5, which may be activated by flagellin, or may include TLR-7, which may be activated by imiquimod. TLR-8 or TLR-9 may likewise be used, which can be activated by resmiquimod or CpG DNA, respectively.

In other examples regarding the stimulator of antigen presentation, such a compound can be added that is capable of upregulating expression of costimulatory molecules on antigen presenting cells. The costimulatory molecules can be selected from CD40, CD80, and/or CD86, for example. The compound capable of upregulating expression of the costimulatory molecules can be an activator of the JAK-STAT pathway, NF-kappa B, NF-kappa B as an inhibitor of i-kappa B, and/or NF-kappa B as an activator of a Pathogen Associated Molecular Pattern (PAMP) receptor, e.g., MDA5, RIG-1, and/or NOD, for example.

In further detail, the compound capable of activating antigen presentation locally can be a dendritic cell, which may be activated with TLR agonist and/or PAMP agonist, or may be generated from patient monocytes, for example. The dendritic cells may likewise be: generated from patient monocytes, autologous to the patient in need of treatment, allogeneic to the patient in need of treatment, activated in vivo by administration of GM-CSF, and/or activated in vivo by administration of FLT-3L, for example.

With respect to examples where there is induction of (at least partial) localized tumor death, this may be achieved by administration of: localized radiation therapy, cryoablation, hyperthermia, and/or localized administration of chemotherapy. If chemotherapy is used, the chemotherapy can be selected from administration of acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-n1, interferon alfa-n3, interferon β-I a, interferon γ-1 b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicarnycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, and/or zorubicin hydrochloride, for example.

In some examples, a method of treating cancer can include administering a lysate-based cancer telocyte vaccine to a patient with cancer to induce an immune response against the cancer, wherein the lysate-based cancer telocyte vaccine includes telocytes from cancer tissue. The telocytes from the cancer tissue can be expanded under conditions resembling the tumor microenvironment. In some examples, the patient is also afflicted with immune suppression. The lysate-based cancer telocyte vaccine can augment antigen presentation of cancer associated antigens from the cancer. The method can also include administering a therapeutic capable of reducing tumor-associated immune suppression, e.g. surgery, chemotherapy, and/or radiation, etc.

As the present disclosure relates to the disruption of telocyte activity, the terms “disruption,” “disrupting,” or the like are used broadly herein as they relate to the activity or function of telocytes. The disruption of telocyte activity in the present disclosure refers specifically to the delivery of a compound or compounds to a subject, either systemically or locally at a specific tissue or organ site, and the delivery of that compound or compounds disrupts the telocyte activity. The term “activity” as it relates to telocytes includes any function of a telocyte that is involved in, promotes, or allows neoplasia in tissue and/or organs of any type, whether malignant or benign. For example, disrupting telocyte activity by the delivery of one or more compounds to a subject may include arresting, reducing, regressing, or reversing the activity of telocytes as it relates to intercellular signaling or other interactions between nearby tissue cells (e.g., interfering with regeneration or repair of organs, interfering with electrical and/or chemical signaling, interfering with electrical and/or chemical sensing, etc.), as well as intracellular processes within the telocyte cell (e.g., interfering with cellular functions related to telocyte cellular health, which may include cellular death).

In one example, the disruption of telocyte activity may be obtained by targeting cells expressing a telocyte-specific marker(s). As an example, the targeting of telocyte cells that express vimentin (a structural protein encoded with VIM gene and a type II intermediate filament protein expressed in mesenchymal cells) and CD34 (a transmembrane phosphoglycoprotein encoded by the CD34 gene) can be carried out using a bispecific antibody or protein to inhibit telocytes.

In another example, disrupting telocyte activity can be performed using a compound (delivered in a composition or agent) that blocks telocyte oncogenic signaling related to the formation of tumors and/or blocks telocyte angiogentic signaling related to the formation of blood vessels. By blocking or reducing the signaling of the telocytes between tissue cells and/or cells of adjacent tissues, information related to neoplasia that may be delivered from one cell to another may be ameliorated. For example, signaling may be inhibited using PDGF inhibitors, Ras/ERK2 inhibitors, miR-942-3p inhibitors, and/or MMP9 inhibitors.

In other examples, telocytes may be used to prepare vaccines to generate an immune response against neoplasia, including cancerous cells and tumors. The immune system has been leveraged thought vaccination to combat traditional pathogens, such as bacterial, parasitic, and viral infections. There is some debate as to whether cancers could be rejected effectively by the immune system since cancer cells are not foreign (like viral infections), but rather such cancer cells are derived from the host. Though derived from the host, cancer cells are abnormal, as indicated by increased proliferation, lack of differentiation, etc. By initiating an anticancer immune response, a patient or subject could thus recover from the disease without the need for surgery, chemotherapy, and/or radiation. This is possible by targeting certain immunologically recognizable markers that are specific to cancer cells.

In accordance with this, the present disclosure relates to the delivery of compounds or combinations of compounds to a subject with cellular targets for the treatment of cancer cells, cancerous neoplasia, or other neoplasia. Cancerous cells or cancerous neoplasms/tumors can be in the form of breast cancer, melanoma, prostate cancer, colon cancer, multiple myeloma, lymphoma, pancreatic cancer, cervical cancer, leukemia, carcinoma, heart tumors, thyroid cancer, retinoblastoma, esophageal cancer, stomach cancer, bladder cancer, etc. Treatment of benign neoplasms/tumors can be for the treatment of tumors in the form of lipoma, meningioma brain tumor, bone tumor, pheochromocytoma, neuroma, teratoma, papilloma, tumors from tuberous sclerosis, etc. The cellular target(s) that receive the delivered compound(s) result in disruption of the activity of telocytes associated with the cancerous cells and/or neoplasm(s), either by pathway interruption due to the introduction of the compound(s) or by immunity response due to the introduction of the compound(s).

Suppression of telocyte activity or viability can be achieved by inhibition of molecular pathways, such as by inhibiting pathways of telocytes associated with cancer. In some examples, the MAPK pathway of telocytes may be inhibited, such as by inhibition of associated pathways including the ERK and/or miR-942-3p/MMP9 axis. Inhibitors of the ERK pathway by any of a number of methodologies available for the suppression of activity or gene expression can be used. To illustrate, induction of RNA interference can be carried out. For example, siRNA and/or shRNA may be developed targeting ERK and/or MMP9 and/or miR-942-3p. ERK is a subfamily of MAPK that is activated by EGF (Epidermal Growth Factor), serum-stimulation, or oxidative stress. ERK is divided into ERK1/2, ERK5, ERK7, ERK8 based on differences in the signal transduction pathways in which they are involved. Ligand-binding to tyrosine kinase receptors, such as the epidermal growth factor receptor (EGFR), results in signal flow, which phosphorylates and activates TEY-motifs present in the activation loops of ERK.

The culture conditions used that are capable of inhibiting ERK are not particularly limited, and any of a number of compounds can be used in a culture composition or in a series of culture compositions. For example, a culture can be used that includes a compound to inhibit the activity of ERK, such as an ERK signal inhibitor (e.g., anti-ERK antibody, enzyme inhibitor involved in activation of ERK, ERK kinase, etc.). Thus, any ERK inhibitor in the form of a compound(s), culture, composition, etc., can be used as long as it acts to inhibit ERK in some way to disrupt the activity of the telocytes being targeted. Examples of ERK inhibitors that can be used include PD0325901, Olomoucine, Aminopurvalanol A, AS703026, AZD8330, BIX02118, BIX02189, CI-1040, Cobimetirlib, GDCs-0623, MEK162, PD318088, PD98059, Refametinib, to name a few.

In other examples, cellular targets that receive the delivered compound(s) can also result in the generation of an immunological response that ultimately acts to disrupt the activity of the telocytes associated with the cancerous cells and/or neoplasm(s). Thus, the result can be the inactivation of cancer cells that are associated with telocytes. In one embodiment, a population of immune cells can be elicited towards cancer telocytes and/or cancer telocyte specific antigens. These immune cells can be generated either in vivo by administration of cancer telocytes and/or cancer telocyte derived components, or generated ex vivo by culture of immune cells with autologous or allogeneic telocytes. In some more specific examples, CD4 and/or CD8 T cells can be generated from a patient by culturing patient T cells ex vivo with cancer telocytes and/or cancer telocyte specific antigens. In some examples, dendritic cells (DCs) can also be used to promote an immune response.

In some examples, polyvalent vaccines with other cellular therapies as an initial poly-immunogenic composition is described. For example, cellular lysates of telocyte cells extracted from tumors and/or generated to possess a “tumor telocyte” phenotype can be loaded into dendritic cells. In other examples, generating a population of cells with tumoricidal ability that are polyvalently reactive can be provided, to which focus is added by subsequent peptide specific vaccination. Cytotoxic lymphocytes may be generated, for example, by extracting peripheral blood from a cancer patient and peripheral blood monoclear cells (PBMCs) can be isolated using any of a number of isolation methods, e.g., the Ficoll method. The PBMCs may be subsequently re-suspended in a serum free-medium, such as AIM-V medium, and allowed to adhere onto a plastic surface over a short period of time, e.g., 2-4 hours. The adherent cells can then be cultured, e.g., 37° C. in AIM-V media supplemented with 1,000 U/mL granulocyte-monocyte colony-stimulating factor and 500 U/mL IL-4, after non-adherent cells are removed by gentle washing in Hanks Buffered Saline Solution (HBSS). Volumes of the supplemented media culture, e.g., about half, can be changed periodically, e.g., every other day. An immature dendritic cell (DC) or dendritic cells (DCs) may be harvested after a period of days, e.g., 7 days. In one example, generated dendritic cells can be used to stimulate T cell and NK cell telocyte killing activity by pulsing with autologous or allogeneic telocyte lysate. Specifically, generated dendritic cells may be further purified from culture through use of flow cytometry sorting or magnetic activated cell sorting (MACS), or may be utilized as a semi-pure population. Dendritic cells pulsed with telocyte lysate may be administered to a patient in need of therapy to stimulate NK and T cell activity in vivo, or in another embodiment, may be incubated in vitro with a population of cells containing T cells and/or NK cells. In some examples, dendritic cells can be exposed to compounds capable of stimulating maturation in vitro and rendering them resistant to tumor derived inhibitory compounds, such as arginase byproducts. Specific ways of stimulating in vitro maturation include culturing dendritic cells or dendritic cells containing populations with a toll-like receptor agonist. Another way of achieving dendritic cell maturation involves exposure of dendritic cells to TNF-α, e.g., at a concentration of approximately 20 ng/mL. In order to activate T cells and/or NK cells in vitro, cells can be cultured in media containing interferon γ, e.g., approximately 1000 IU/mL. Incubation with interferon γ may be performed for a period from about 5 hours to about 7 days, for example. In one embodiment, the period of incubation may be about 24 hours, after which T cells and/or NK cells are stimulated via the CD3 and CD28 receptors. One way of accomplishing this is by adding antibodies capable of activating these receptors. In one embodiment, anti-CD3 antibody, e.g., approximately 2 μg/mL may be added with anti-CD28, e.g., about 1 μg/mL. In order to promote survival of T cells and NK cells, as well as to stimulate proliferation, a T cell/NK mitogen may be used. In one embodiment, the cytokine IL-2 may be utilized, e.g., 500 u/mL Media containing IL-2 and antibodies may be changed periodically, e.g., every 48 hours for approximately 8-14 days. In one particular embodiment, dendritic cells are included with the T cells and/or NK cells in order to endow cytotoxic activity towards telocyte cells. In some examples, inhibitors of caspases can be added in the culture so as to reduce the rate of apoptosis of T cells and/or NK cells. Generated cells can be administered to a subject through any traditional route of administration, such as intra-dermally, intramuscularly, subcutaneously, intraperitoneally, intra-arterially, intravenously (including a method performed by an indwelling catheter), intra-tumorally, or into an afferent lymph vessel. The immune response of the patient treated with these cytotoxic cells can be assessed utilizing a variety of antigens found in tumor cells. When cytotoxicity, antibody, or antibody associated with complement fixation are recognized to be upregulated in the cancer patient, subsequent immunization(s) can be performed utilizing peptides to induce a focusing of the immune response.

Utilizing dendritic cells to stimulate immunity has been validated in animal studies taking advantage of the ability of immature dendritic cells to potently phagocytose various antigens. If the antigens possessed DAMPs, or if DAMPs were present in the environment, the dendritic cells would mature and present the antigens, resulting in stimulation of potent T cell immunity. Accordingly, in the initial studies, immature dendritic cells were incubated with various antigens, and then a maturation signal (replicating natural DAMPs) was applied and the dendritic cells were injected into animals. Thus, dendritic cells may be utilized as a type of “cellular adjuvant.” Classical adjuvants, such as Fruend's Adjuvant, contained a high concentration of DAMPs, which resulted in the stimulation of local dendritic cells at vaccination site in vivo.

Dendritic cells have been studied clinically for use against prostate cancer. There, thirty three androgen resistant metastatic prostate cancer patients were treated with dendritic cells that were pulsed with peptides from a prostate specific antigen termed PMSA. Nine partial responders were identified based on NCPC criteria, plus 50% reduction of PSA. Four of the partial responders were also responders in the Phase I study, with an average response duration of 225 days. Their combined average total response period was over 370 days. Five other responders in the secondary immunizations at the Phase II study were non-responders in the Phase I study. Their average partial response period was 196 days. Later, thirty-three additional patients that had not received prior dendritic cell immunization in the Phase I study were evaluated. All of these subjects received six infusions of dendritic cells pulsed with PSM-P1 and -P2 at six week intervals without any treatment associated adverse events. Six partial and two complete responders were identified in the Phase 11 study based on NPCP criteria, plus 50% reduction of prostate-specific antigen (PSA), or resolution in previously measurable lesions on a ProstaScint scan. The same group analyzed immune response in patients who had clinical remission or relapsed. A strong correlation was found between delayed type hypersensitivity response to the PSM-P1 and PSM-P2 and clinical response.

Another subsequent study utilized dendritic cells generated using GM-CSF and IL-4 but pulsed with PAP, which is another prostate antigen. Specifically, the PAP was delivered to the dendritic cells by means of generation of a PAP-GM-CSF fusion protein. Two intravenous infusions of the generated cells were performed one month apart in 12 patients with androgen resistant prostate cancer. The infusions were followed by three subcutaneous monthly doses of the fusion protein without cells. Treatment was well tolerated and circulating prostate-specific antigen levels dropped in three patients. Immune response to the fusion protein was observed, as well as to PAP. In addition to prostate cancer, in which FDA approval has been granted for the Provenge drug, numerous trials have been conducted in a wide variety of cancers. All the trials demonstrated safety, without serious adverse effects, of dendritic cell administration, as well as some degree of therapeutic efficacy. In further detail, trials have been conducted in melanoma, soft tissue sarcoma, thyroid, glioma multiple myeloma, lymphoma, leukemia, as well as liver, lung, ovarian, and pancreatic cancer. The data of these studies support the safety of follow-up infusion of dendritic cells that have been pulsed with tumor antigen derived peptide.

Furthermore, T cell activation can be performed in vivo. T cells can be immune effectors against tumors, possessing ability to directly kill via CD8 cytotoxic cells, or indirectly kill tumors by activation of macrophages through interferon γ production. Additionally, T cells have been shown to convert protumor M2 macrophages to M1. T cells in the context of cancer may be illustrated by a positive correlation between tumor infiltrating lymphocytes and patient survival. In addition, positive correlations between responses to various immunotherapies has been made with tumor infiltrating lymphocyte density. Increased T cell activity is associated with reduction in T regulatory (T_{reg}) cells. Studies show that using compounds that cause suppression of T_{reg} cells correlates with improved tumor control. Compounds that inhibit T_{reg} cells include arsenic trioxide, cyclophosphamide, triptolide, gemcitabine, and artemether.

Returning to promoting an immunity response to cancer as it relates to telocytes (rather than more traditional cancer cells previously studied), it is noted that telocytes can be utilized to enhance activity of other immunotherapies such as CAR-T cells, NK cells and targeting chemotherapy and/or immunotherapy. In other examples, the immunization with cancer telocytes can be used to enhance efficacy of other antigen specific immunotherapies. In still other embodiments, immunization with cancer telocytes increases responses to polyvalent tumor vaccine such as CanVaxin, or other polyvalent vaccine mixtures, Immunity to telocytes in accordance with the present disclosure can increase efficacy of vaccination or cell therapy targeting tumor antigens. Example cell therapy targeting tumor antigens include ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, carbonic anhydrase IX, PSCA, Legumain, or MAD-CT-1 (protamine 2).

In other embodiments, dendritic cells are generated from leukocytes of patients by leukopheresis. Numerous ways of promoting leukopheresis can be used, including a COM.TEC®, from Fresenius Kabi AG (Germany), which can be utilized with the Fresenius COM.TEC MNC program, e.g., 1500 rpm using a P1Y kit and plasma pump flow rates adjusted to approximately 50 mL/min. Various anticoagulants may be used, but in one example, the anticoagulant can be ACD-A. The Inlet/ACD Ratio may be ranged from approximately 10:1 to 16:1, for example, based on target anticoagulation. In one embodiment, approximately 150 mL of blood is processed, though other volumes can be similarly processed. The leukopheresis product is subsequently used for initiation of dendritic cell culture. In order to generate peripheral blood mononuclear cells from leukopheresis product, mononuclear cells can be isolated by the Ficoll-Hypaque density gradient centrifugation. Monocytes can then be enriched by the Percoll hyperosmotic density gradient centrifugation followed by two hours of adherence to the plate culture. Cells can then be centrifuged, e.g., 500×g, to separate the different cell populations. Adherent monocytes can be cultured for a period of days, e.g., about 7 days, and separated in multiple well plates, e.g., 6-well plates at 2×106 cells/mL RMPI medium with 1% (v/v) penicillin/streptomycin, 2 mM L-glutamine, 10% (v/v) of autologous, 50 ng/mL GM-CSF, and 30 ng/mL IL-4. After several days, e.g., day 6, immature dendritic cells are pulsed with tumor antigen to form hybrid cells or hybridomas, e.g., via incubation of lysates with dendritic cells, or a cellular fusion product may be generated by fusion of immature dendritic cells with tumor cells by treatment with polyethylene glycol. Later, e.g., day 7, immature dendritic cells can then induced to differentiate into mature dendritic cells by culturing, e.g., 48 hours with 30 ng/mL interferon γ (IFN-γ). During the course of generating dendritic cells for clinical purposes, microbiologic monitoring tests are performed at the beginning of the culture, e.g., day 5, and at the time of cell freezing for further use or prior to release of the dendritic cells. Administration of tumor pulsed dendritic cells is utilized as a polyvalent vaccine, whereas subsequent to administration, antibody or t cell responses are assessed for induction of antigen specificity, and peptides corresponding to immune response stimulated are used for further immunization to focus the immune response.

In other examples, a culture of immune effector cells can be performed after extracting from a patient that has been immunized with a polyvalent antigenic preparation. Specifically, separating a cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker(s) used for the isolation may include CD3, CD8, and/or CD4. Separation methods can be carried out, depending on these surface markers, such as by any of a number of methodologies. In one example, isolation can be performed by mixing a carrier, e.g., beads or a culturing container on which an anti-CD8 antibody has been immobilized, with a cell population containing T cells, followed by recovering CD8-positive T cells bound to the carrier. Beads on which an anti-CD8 antibody has been immobilized, e.g., CD8 MicroBeads, Dynabeads M450 CD8, Eligix anti-CD8 mAb coated nickel particles, etc., can be suitably used. This same procedure can be used when implementing CD 4 as an index, using beads such as CD4 MicroBeads, Dynabeads M-450, etc. In some embodiments, T regulatory cells may be depleted before initiation of the culture. Depletion of T regulatory cells may be performed by negative selection by removing cells that express makers such as neuropilin, CD25, CD4, CTLA4, and membrane bound TGF-β. Different culture conditions in order to generate effector lymphocytes or cytotoxic cells that possess both maximal activity in terms of tumor killing as well as migration to the site of the tumor can likewise be used as determined by experimentation. For example, culturing a cell population and cell sub-population containing a T cell can be performed by selecting suitable known culturing conditions depending on the cell population. In addition, stimulating a cell population may be carried out by adding proteins and/or chemical ingredients known for stimulating target cell populations to the medium to perform culturing. For example, cytokines, chemokines or other ingredients may be added to the medium.

The cytokines, chemokines, and other ingredients used for stimulating the cell population are not particularly limited, provided they can act on the T cell. Cytokines that may be used include IL-2, IFN-γ, transforming growth factor (TGF)-β, IL-15, IL-7, IFN-α IL-12, CD40L, or IL-27, for example. From the viewpoint of enhancing cellular immunity, IL-2, IFN-γ, or IL-12 may be particularly suitable. From the viewpoint of improvement in survival of a transferred T cell in vivo, IL-7, IL-15 or IL-21 may be particularly useful. Furthermore, the use of various chemokines is not particularly limited, provided the chemokines act on the T cell and exhibit migration activity. Examples of chemokines that can be used include RANTES, CCL21, MIP1α, MIP1β, CCL19, CXCL12, IP-10, or MIG.

The stimulation of a cell population can likewise be carried out in the presence of a ligand for a molecule present on the surface of the T cell, e.g., CD3, CD28, CD44 and/or an antibody to the molecule. In other examples, a cell population can be stimulated by contacting with other lymphocytes such as antigen presenting cells, such as dendritic cells, and presenting a target peptide, e.g., a peptide derived from a cancer antigen on the surface of a cell.

The present disclosure is also drawn to assessing cytotoxicity and migration as end points, and also formulating a cellular product having enhanced function based on other ways of assessing T cell activity. For example, the cellular product can be optimized for assessing T cell activity. This enhanced function of the T cell can be assessed at multiple time points before and after individual steps, e.g., before and after each step, using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer, a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method.

In vivo assessment of the efficacy of generated cells may be assessed in a living body before first administration of the T cell with enhanced function of, or at various time points after initiation of treatment using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of additional methods for measuring an increase in an immune response may include the use of a delayed hypersensitivity test, flow cytometry using a peptide major histocompatibility gene complex tetramer, a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method.

An immune response can be assessed by a weight, diameter, or malignant degree of a tumor possessed by a living body, or may be assessed by the survival rate or survival term of a subject or group of subjects. The cells can be expanded in the presence of specific antigens associated with tumors and subsequently injected into the patient in need of treatment. Expansion with specific antigens may be carried out by co-culture with proteins selected from: a) ROBO; b) VEGF-R2; c) FGF-R: d) CD105; e) TEM-1; or f) surviving, for example, as well as with any other proteins that may be similarly effective for expansion.

In other examples, an injectable polysaccharide purified from potato sprouts can be administered systemically and/or locally. For example, ImmunoMax® from Avexima (Russia) is an approved pharmaceutical in the Russian Federation (registration P No. 001919/02-2002) and 5 other countries of the Commonwealth of Independent States (formerly the USSR) and has been evaluated in a wide range of medical situations. In accordance with the formal “Instruction of Medical Use” of ImmunoMax®, one medical indication is the stimulation of immune defense during the treatment of infectious diseases, and may promote the immune-mediated killing of cancer cells in a TLR4-dependent manner. Thus, in one example of the present disclosure, ImmunoMax® can be utilized to induce an M2 to M1 shift, thus reducing macrophage derived immune suppressants and augmenting production of immune stimulatory cytokines such as IL-12 and TNF-α. In some examples, other compounds may likewise be used to modulate M2 to M1 transition of tumor associated macrophages including RRx-001, such as bee venom derived peptide melittin, CpG DNA, metformin, Chinese medicine derivative puerarin, rhubarb derivative emodin, dietary supplement chlorogenic acid, propranolol, poly ICLC, BCG, Agaricus blazei Murill mushroom extract, endotoxin, olive skin derivative maslinic acid, intravenous immunoglobulin, phosphotidylserine targeting antibodies, dimethyl sulfoxide, surfactant protein A, Zoledronic acid, or bacteriophages, for example.

Prior to induction of immunogenic cell death, antigen presenting cells can be administered, and one of the most potent antigen presenting cells is the dendritic cell. Dendritic cells possess unique morphology similar to neuronal dendrites and were originally identified based on their ability to stimulate the adaptive immune system. In the field of tumor immunotherapy, dendritic cells appear to be unique in their ability in the body to activate naïve T cells. The concept of dendritic cells instructing naïve T cells to differentiate into effector or memory cells places the dendritic cell as a powerful antigen presenting cell. Thus, for immunotherapeutic purposes, dendritic cells do not necessarily need to be administered at high numbers to patients. Dendritic cells have been described as sentinels of the immune system that are patrolling the body in an immature state, and once activated by a stimulatory signal such as a Damage Associated Molecular Patterns (DAMPS), the dendritic cells migrate into the draining lymph nodes through the afferent lymphatics. During the trafficking process, dendritic cells degrade ingested proteins into peptides that bind to both MHC class I molecules and MHC class II molecules. This allows the dendritic cells to: a) perform cross presentation in that they ingest exogenous antigens but present peptides in the MHC I pathway; or b) activate both CD8 (via MHC I) and CD4 (via MHC II), unlike lipid antigens that are processed via different pathways and are loaded onto non-classical MHC molecules of the CD1 family.

### Definitions

Unless defined otherwise, all technical and scientific terms, terms of art, and acronyms used herein have the meanings commonly understood by one of ordinary skill in the art in the field(s) of the invention, or in the field(s) where the term is used.

As used herein, a plurality of elements, compositional components, and/or materials may be presented in a common list for convenience. However, these lists should be construed as though individual members of the list are individually identified as separate and unique members. Thus, no individual member of such list should be construed as a de facto equivalent of any other member of the same list solely based on presentation in a common group without indications to the contrary.

It is also understood that terms used herein will take on the ordinary meaning in the relevant technical field unless specified otherwise. In some instances, there are terms defined more specifically throughout the specification or included below, and thus, these more specifically defined terms have a meaning as defined, unless the context clearly dictates otherwise.

The singular forms “a” “an,” and “the” include plural references unless the context clearly dictates otherwise. Thus, for example, reference to “a cell” includes reference to one or more cells.

As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Furthermore, the term “about” expressly includes the exact range parameters set forth in the range that uses “about.” Thus, about 1 to about 5 may allow for minimal flexibility of range parameters, but also directly supports the exact range of from 1 to 5.

As used herein, “comprising” or “including” language or other open-ended language can be substituted with “consisting essentially of” and “consisting of” as if such transition phrase is expressly included in such embodiments.

The term “example(s)” or “embodiment(s),” particularly when followed by a listing of terms, concentrations, components or ingredients of a composition, method steps, etc., is merely exemplary and illustrative and should not be deemed to be exclusive or comprehensive.

“Adjuvant” refers to a substance that is capable of enhancing, accelerating, or prolonging an immune response when given with a vaccine immunogen.

“Agonist” refers to a substance which promotes (induces, causes, enhances or increases) the activity of another molecule or a receptor. The term agonist encompasses substances which bind a receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more compounds to the same subject during a treatment period. The two or more compounds may be encompassed in a single formulation and thus be administered simultaneously. Alternatively, the two or more compounds may be in separate physical formulations and administered separately, either sequentially or simultaneously to the subject. The term “administered simultaneously” or “simultaneous administration” means that the administration of the first compound and that of a second compound overlap in time with each other, while the term “administered sequentially” or “sequential administration” means that the administration of the first compound and that of a second compound does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen such as an immunogenic polypeptide) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

As used herein, the terms “treatment” or “treating” of a condition and/or a disease in a subject, such as a human, includes preventing a disease or a condition related to a disease, e.g., avoiding any clinical symptoms of the disease; inhibiting the condition or disease, e.g., arresting or slowing the development or progression of the disease or disease symptoms; and/or relieving or ameliorating symptoms of the disease, e.g., causing the regression of clinical symptoms and/or retreat of the disease. In many instances, the disease may be a cancer. Treatment also includes use of compositions of the present disclosure alone or as a combination of compounds (or combination of compositions) via administration by any of a number of delivery routes before, during, or after diagnosis of a disease. In some instances, treatment may include administration for the prevention of a disease for at risk individuals or across a population. Treatment may include a single treatment or multiple treatments in series. Treatment routes of delivery can include intravenous, e.g., vascular injection or infusion, intramuscular, nasal, oral (e.g., swallowing), ocular, mucosal (e.g., sublingual, buccal, nasal, anal, vaginal, etc.), cutaneous (e.g., topical), subcutaneous, transdermal, inhalation, or any other administration route suitable for delivery to a subject's tissue or organ site impacted by the disease being treated.

“Treating a cancer,” “inhibiting cancer,” “reducing cancer growth,” etc., refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. In alignment with this, the terms “cancer”, “cancer cell” “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention. Although the word “lymphocyte” is used, this also includes heterogeneous cells that have been expanded during the ex vivo culturing process including dendritic cells, NKT cells, γ delta T cells, and various other innate and adaptive immune cells.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” refers to progressive, malignant diseases of the hematopoietic organs/systems and is generally characterized by a distorted proliferation and development of leukocytes and their precursors in the blood and bone marrow. Leukemia diseases include, for example, acute non-lymphocytic leukemia, chronic lymphocytic leukemia, acute granulocytic leukemia, chronic granulocytic leukemia, acute promyelocytic leukemia, adult T cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, or promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. The term “polypeptide” is used interchangeably with “peptide”, “altered peptide ligand”, and “flourocarbonated peptides.” The term “pharmaceutically acceptable carrier” includes any and all solvents, dispersion media, coatings, antibacterial and antifungal compounds, isotonic and absorption delaying compounds, and the like. The use of such media and compounds for pharmaceutically active substances is well known in the art.

Except insofar as any conventional media or compound is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an αβ T cell expressing TCR α and β chains, and a γδ T cell expressing TCR γ and δ chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo, in vitro (ex vivo). These cells include any cells collected from a living body, or cells obtained via ex vivo culture, for example, a T cell population obtained by the method of the present invention as it is, or obtained by freeze preservation. The term “antibody” is meant to include both intact molecules as well as fragments thereof that include the antigen-binding site. Whole antibody structure is often given as H.sub.2L.sub.2 and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contain the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered. The terms “individual”, “host”, “subject”, and “patient” are used interchangeably herein, and refer to a mammal, including, but not limited to, primates, for example, human beings, as well as rodents, such as mice and rats, and other laboratory animals.

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein the treatment or method comprises administering to an animal, such as a mammal, especially a human being, a sufficient amount of two or more chemical compounds or components of the regimen to effectively treat a disease or to produce the physiological change, wherein the chemical compounds or components are administered together, such as part of the same composition, or administered separately and independently at the same time or at different times (i.e., administration of each compound or component is separated by a finite period of time from one or more of the compounds or components) and where administration of the one or more compounds or components achieves a result greater than that of any of the compounds or components when administered alone or in isolation.

The term “anergy” includes failure to react to an antigen or unresponsiveness of an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”. “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

It is also noted that when discussing the various compositions and treatment methods of the present disclosure, these discussions can be considered applicable to one another whether or not they are explicitly discussed in the context of that example. Thus, for example, when discussing treatment of adenocarcinoma related to lung cancer, such disclosure is also relevant to and directly supported in the context of the other adenocarcinomas, such as those found in the breast, prostate, colon, etc., or even other types of cancers in contexts where similar mechanisms may be present.

### Examples

The following examples illustrate embodiments of the disclosure. However, it is to be understood that the following are only exemplary or illustrative of the application of the principles of the present technology. Numerous modifications and alternative compositions, methods and/or systems may be devised without departing from the scope of the present disclosure. The appended claims are intended to cover such modifications and arrangements. Thus, while the present disclosure has been described above with particularity, the following examples provide further detail in connection with what are presently deemed to be practical embodiments of the disclosure.

### Example 1—Extraction of Telocytes from Tumor Tissue

To isolate telocytes from tumor tissue, about 0.2 to 5 grams of tumor tissue obtained is minced and treated with 2.5 mL of DMEM (Dulbecco's Modified Eagle Medium, from Thermofisher) supplemented with and 0.1% (v/v) trypsin/TrypLE. The treated tumor tissue is incubated for 10 min at 37° C. on a shaker (180 rpm). A supernatant is then removed, and then collagenase and additional trypsin/TrypLE medium (1 mL of 025% (v/v) Trypsin/2 mL, of TrypIE per 50 mg of tissue are added. The mixture is then incubated again, this time at 45 minutes at 37° C. on the shaker (180 rpm). The digested tissue is dissociated by gentle pipetting away every 15 min.

The supernatant that is removed is sequentially filtered through a 100 μm and a 41 μm nylon mesh, and then a cell suspension collected is centrifuged at 50×g (relative centrifugal force of centrifuge tubes to the machine, e.g. 50:1 ratio) for 2 min using a Beckman centrifuge. The supernatant is then removed and re-centrifuged at 300×g for 10 min. The pellet is re-suspended in 5 mL of PEB medium (PBS supplemented with 0.5% (v/v) bovine serum albumin and 2 mM EDTA [pH 7.2]). The mixture is then centrifuged at 38×g for 2 min to remove the debris, and the collected supernatant is further centrifuged at 200×g for 10 min. The cell pellet is then mixed with 1 mL of PEB medium and 5 μL of a rabbit anti-rat C-kit antibody, and then the sample is incubated at 4° C. for 40 min.

An additional 2 mL of PEB medium is added, and the mixture is centrifuged at 458×g for 4 min to collect the cells. The pellet is re-suspended in 160 μL of PEB medium, and 20 μL of a solution containing goat anti-rabbit IgG microbeads is added, followed by incubation at 4° C. for 25 min. The mixture is then added to a magnetic separation (MS) column in a magnetic field, and the unlabeled cells are allowed to pass through. The MS column is then removed from the magnetic field, and the labeled cells are flushed out with additional PEB medium.

An isolated cell pellet is collected by centrifugation at 458×g for 4 min, followed by culture in DMEM containing 20% (v/v) fetal calf serum at 37° C. and 5% CO₂ in a 95% (v/v) air incubator. The isolated cell pellet of telocytes from the tumor cells is about 100,000 to 1,500,000 cells are obtained.

### Example 2—Expanding Telocytes Ex Vivo in Replicated Tumor Microenvironments

Replication of cancer microenvironment for growth of cancer associated telocytes is carried out under one or more of a number of conditions.
A) Replication of a cancer microenvironment is carried out under conditions of hypoxia generated at a level approximately equal to the amount which induces translocation of HIF-1 α into the tumor. This can be carried out, for example, by tri-gas incubator controlling nitrogen, oxygen and carbon dioxide. Traditional cell culture uses on only oxygen and carbon dioxide for normomoxia-oxygen levels of 21% similar to room air. However, using the tri-gas, the nitrogen is increased to allow the oxygen level to decrease to 5% (v/v) in the incubator for at least 24 hours at 37 degrees Celsius. This allows a true hypoxic environment similar to the in-vivo tumor environment. Telocytes (1,000,000) are isolated from human lung cancer biopsy of 5 gm and placed in the tri-gas environment with only 5% (v/v) oxygen at 37° C. for 24 hours. At 24 hours 260,000 telocytes are present and had a 6× increased expression of HIF-1 α compared to nor c c
B) Replication of a cancer microenvironment is carried out using cytokines and other factors associated with tumor microenvironments. Suitable cytokines for use include IL-10, soluble HLA-G, TGF-β, arginase, leptin, LIF, PGE2, galectin 1, galectin 2, galectin 3, galectin 7, galecin-9, or siglec-5, along with conditioned media derived from cancer cells growing either in two dimensional or three dimensional culture. Fresh lung cancer tissue (5 gm) is obtained and cut into smaller pieces, and incubated with 5 mg/ml collagenase type II (v/v) (Sigma-Aldrich, St. Louis, MO, USA) for 10 min. Then, samples are washed twice with calcium- and magnesium-free PBS (Servicebio, USA) and centrifuged at 10000 r.p.m. for 5 min, and then resuspended in DMEM (Gibco, NY, USA) supplemented with 10% fetal calf serum (v/v). Using 250 magnification of the microscope and transferring cells to 0.2 mL centrifuge tubes containing 2 μL of lysate according to the morphology of telocytes.
C) Lung cancer conditioned media with cells are first plated (1×106 cells) and cultured for 48 hours as previously described. The conditioned media is removed and filtered prior to use. The lung cancer cells are similarly cultured in RPMI+10% FBS, with penicillin-streptomycin. Sorted healthy donor CD14+ monocytes are plated in R10% or with 100% of total volume telocyte cultured media or 10 ng/ml of human recombinant cytokines (PeproTech: TGF-β; IL-6; IL-10; G-CSF and VEGF) in 24 well plates for 48 h. These monocytes are harvested and used for DC generation.

### Example 3—Preparation of Imrnortalized Telocytes for Cancer Vaccine

A) Primary human lung cancer telocytes prepared as described in any of Example 2 are immortalized by transduction with SV40 small and large T antigen. Transduction is performed using lentivirus. The transfer plasmid pCMV SV40. Primary cells are seeded at a density of 10,000 cells/cm²; 24 hours post-seeding cells are transfected with lentivirus at a multiplicity of infection (MOI) of 5. Proliferation is monitored using the Incucyte Live Cell Imaging System. After passaging, cells are seeded at a very low density (1-2 cells/cm²) in 10 cm Petri dishes. Individual and well-delimited clones are picked and transferred into 96 well plates and frozen at passages 7-10.
B) Another approach to immortalization is using the primary human lung cancer telocytes where the cells are sub-cultured onto six-well dishes and allowed to recover for 4 days. Cells are then infected for 16 h with the hTERT retroviral vectors in the presence of polybrene (4 μg/ml) and selected in puromycin (600 ng/ml) for 4 wk. After to transfection with the virus, puromycin is used to kill all non-infected cells to allow colony formation of the hTERT-infected cells. Cell lines are generated by clonal selection and are maintained in DME/F12 medium with 10% FBS and antibiotic/antimycotic (10000 units/ml) (v/v). Stable lung cancer telocyte hTERT lines have now had 40 passages and over 250 doublings without karyotype instability.

### Example 4—Preparation of Cytotoxic Lymphocytes and Harvesting Immature Dendritic Cells

Cytotoxic lymphocytes are generated by extracting 50 mL of peripheral blood from a cancer patient and peripheral blood monoclear cells (PBMCs) are isolated using the Ficoll method. The PBMCs are subsequently re-suspended in a 10 mL or AIM-V medium and allowed to adhere onto a plastic surface over 2-4 hours. The adherent cells are cultured at 37° C. in the AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day, and immature dendritic cells are harvested on day 7. The generated dendritic cells may be further purified from culture through use of flow cytometry sorting or magnetic activated cell sorting (MACS), or may be utilized as a semi-pure population.

### Example 5—Using Dendritic Cells to Stimulate Telocyte Killing Activity

Dendritic cells in purified or semi-pure form prepared in accordance with Example 4 are used to stimulate T cell and NK cell telocyte killing activity by pulsing with autologous or allogeneic telocyte lysate. Dendritic cells electropulsed with telocyte lysate are used in several different ways, including administration to a patient in need of therapy to stimulate NK and T cell activity in vivo; or incubation in vitro with a population of cells containing T cells and/or NK cells. Other types of fusion may be used, including sonoporation or using polyethylene glycol as a fusion compound.

### Example 6—Stimulating In Vitro Maturation of Dendritic Cells that are Cytotoxic to Telocytes for In Vivo Administration

The dendritic cells in purified or semi-pure form of Example 4 are used by exposing them to compounds capable of stimulating maturation in vitro and rendering them resistant to tumor derived inhibitory compounds, such as arginase byproducts. Specific ways of stimulating in vitro maturation include: culturing dendritic cells with a toll-like receptor agonist; or exposing dendritic cells to TNF-α, e.g., at a concentration of about 20 ng/mL.

To activate T cells and/or NK cells in vitro, cells are cultured in media containing approximately 1000 IU/mL of interferon γ. Incubation with interferon γ is performed for 24 hours, after which T cells and/or NK cells are stimulated via the CD3 and CD28 receptors. 2 μg/mL of anti-CD3 antibody is added along with approximately 1 μg/mL anti-CD28. 500 u/mL Cytokine IL-2 is added as a T cell/NK mitogen to promote survival of T cells and NK cells and to stimulate proliferation. The resultant media containing Cytokine IL-2 and antibodies may be changed every 48 hours for approximately 8-14 days. In a particular embodiment, inhibitors of caspases are added in the culture so as to reduce the rate of apoptosis of T cells and/or NK cells. Dendritic cells are included with the T cells and/or NK cells in order to generate a cell culture endowed with cytotoxic activity towards telocyte cells.

Generated cells of the cell culture are administered to a subject intra-dermally, intramuscularly, subcutaneously, intraperitoneally, intra-arterially, intravenously (including a method performed by an indwelling catheter), intra-tumorally, or into an afferent lymph vessel. The immune response of the patient treated with these cytotoxic cells is assessed utilizing a variety of antigens found in tumor cells. When a cytotoxic antibody or antibody associated with complement fixation are recognized to be upregulated in the cancer patient, subsequent immunization(s) is performed utilizing peptides to induce a focusing of the immune response.

### Example 7—Preparation of Dendritic Cells

Dendritic Cells are generated from leukocytes of patients by leukopheresis using a COM.TEC® device (Fresenius Kabi AG, Germany), which is utilized with the Fresenius COM.TEC MNC program at approximately 1500 RPM using a P1Y kit. The plasma pump flow rates set at approximately 50 mL/min. ACD-A anticoagulant is used and the inlet:ACD-A ratio of the device is ranged from approximately 10:1 to 16:1 of anticoagulation. 150 mL of blood is processed and a leukopheresis product generated is subsequently used for initiation of a dendritic cell culture. In order to generate peripheral blood mononuclear cells from the leukopheresis product, mononuclear cells are isolated by a Ficoll-Hypaque density gradient centrifugation. Monocytes are then enriched by the Percoll hyperosmotic density gradient centrifugation followed by 2 hours of adherence to the plate culture. Cells are then centrifuged at 500×g (relative centrifugal force of centrifuge tubes to the machine, e.g., 500:1 ratio) for 10 min using a Beckman centrifuge to separate the different cell populations. Adherent monocytes are cultured for 7 days in 6-well plates at 2×106 cells/mL RMPI medium with 1% (v/v) penicillin/streptomycin, 2 mM L-glutamine, 10% (v/v) of autologous, 50 ng/mL GM-CSF, and 30 ng/mL IL-4. During the course of generating dendritic cells for clinical purposes, microbiologic monitoring tests are performed at the beginning of the culture, on the 5th day and at the time of cell freezing for further use or prior to release of the dendritic cells.

### Example 8—Preparation of DC Vaccine of Dendritic Cells Pulsed with Telocyte Lysate (TelL-DC)

The dendritic cells prepared in accordance with Example 7 are used with autologous or allogenic telocyte lysate to generate dendritic cells pulsed with telocyte lysate. More specifically, on day 6 of the dendritic cell preparation process, immature dendritic cells are pulsed with telocyte lysate via electrical pulse mediated fusion with incubation of the telocyte lysates with dendritic cells to form hybridomas. On day 7, the immature dendritic cells are then induced to differentiate into mature dendritic cells by culturing for 48 hours with 30 ng/mL interferon γ (IFN-γ).

Example 9—Example Uses of Dendritic Cells Pulsed with Telocyte Lysate (TelL-DC)

The dendritic cells in purified or semi-pure form pulsed with autologous or allogenic telocyte lysate prepared in accordance with Example 8 are used to stimulate T cell and NK cell telocyte killing activity. More specifically, dendritic cells pulsed with telocyte lysate are used by administration to a patient in need of therapy to stimulate NK and T cell activity in vivo, or by incubation in vitro with a population of cells containing T cells and/or NK cells.

### Example 10—Preparation of Comparative DC Vaccine of Dendritic Cells Pulsed with Tumor Lysate (TumL-DC)

Dendritic cells prepared in accordance with Example 7 are used to generate dendritic cells pulsed with tumor lysate (tumor antigen in the form of a lysate). More specifically, on day 6 of the dendritic cell preparation process, immature dendritic cells are fused with tumor cells using polyethylene glycol to form cellular fusion products. On day 7, the immature dendritic cells are then induced to differentiate into mature dendritic cells by culturing for 48 hours with 30 ng/mL interferon γ (IFN-γ).

### Example 11—Example Uses of Comparative DC Vaccine of Dendritic Cells Pulsed with Tumor Lysate (TumL-DC)

The dendritic cells pulsed with tumor lysate prepared in accordance with Example 10 are delivered to patients as a polyvalent vaccine. Subsequent to administration, antibody or cell responses are assessed for induction of antigen specificity, and peptides corresponding to immune response are stimulated and are used for further immunization to focus the immune response.

### Example 12—Treatment Study of Patients with Non-Small Cell Lung Cancer

Administration: A six (6) patient population with Stage 4 non-small cell lung cancer were randomized to receive either TelL-DC Vaccine (telocyte lysate) of Example 8 or TumL-DC (tumor lysate) of Example 10. Specifically, three (3) patients received the TelL-DC Vaccine and three (3) patients received the TumL-DC Vaccine. In each case, the patients received one (1) injection dose of their respective dendritic cell vaccine at seven (7) day intervals ×3. Sterility testing for mycoplasma, gram stain and cultures were performed and verified to be negative in all six patient cell products. The total dose was 100 million cells per injection.

Results: All patients received a successful production of their respective DC vaccine with no issues related to dose or sterility, and all six (6) patients tolerated their three (3) doses well. Table 1 provides the results from the study.

TABLE 1
Patient Data from Treatment Study

| | | Change | | |
|---|---|---|---|---|
| | | in Tumor | | Adverse Events |
| | Type of | Size | Response | 30 days, 90 days, 180 days |
| Patient | Vaccine | (180 days) | (180 days) | related to vaccine |
| 1 | TelL-DC | −50% | partial | Fever, non-specific aches, |
| | | | | none |
| 2 | TumL-DC | +10% | none | Fever, delayed |
| | | | | hypersensitivity reaction, |
| | | | | reaction resolved |
| 3 | TumL-DC | 0% | none | Fever, delayed |
| | | | | hypersensitivity reaction, |
| | | | | reaction resolved |
| 4 | TelL-DC | −60% | partial | None, non-specific aches, |
| | | | | none |
| 5 | TumL-DC | −15% | partial | Fever, delayed |
| | | | | hypersensitivity reaction, |
| | | | | reaction resolved |
| 6 | TelL-DC | −60% | partial | None, non-specific aches, |
| | | | | none |

As can be seen in Table 1, all three (3) patients receiving the telocyte lysate-dendritic cell vaccine (TelL-DC) experienced a reduction in tumor size at 180 days and the adverse events were manageable. The three (3) patients that received the tumor lysate dendritic cell vaccine (TumL-DC) hovered around no change in tumor size, with one patient experiencing slight tumor growth (10%) and one patient experiencing slight tumor reduction (−15%).

### Example 13—T Cell-Mediated Killing of Tumor Cells and CAR-T Cell Activation with T Cells Modified with Chimeric Multi-Antigen Receptors

An in vitro assay was conducted on human lung cancer cells to evaluate the function of the CAR-Ts in the killing of tumor cells and the amount of CAR-T cell activation quantified based on the production of interferon gamma (IFN-γ). T cells modified with chimeric multi-antigen receptors were prepared for the study. More specifically, the modified T cell included a human CD34/vimentin chimeric antigen receptor, with signal peptide, anti-CD34 single-chain variable fragment (scFv) CD34 scFv V_{L}-Vimentin scFv V_{H}-Vimentin scFvV_{L}-CD34 scFv V_{H}, CD8α hinge and transmembrane domain, modified 4-1 BB-CD137 costimulatory signaling domain, and CD3 zeta cytoplasmic region. The chimeric multi-antigen receptor was packed into a T cell using retroviral delivery system. In this study, CD34/vimentin was derived from the human lung cancer telocyte lysates. The T cell-mediated killing of tumor cells and CART-T cell activation data is shown in Tables 2 and 3, respectively.

TABLE 2
T Cell Mediated Killing of Tumor Cells

| | Cell Death (%) from Treatment with Varying | | |
|---|---|---|---|
| CD34/Vimentin | Levels of CD34/Vimentin Expression | | |
| Expression | Control | CAR-T | T Cell |
| None (0%) | 2 | 5 | 7 |
| Low (>0% to 25%) | 5 | 65 | 10 |
| Mid (>25% to 75%) | 1 | 81 | 12 |
| High (>75%) | 6 | 98 | 14 |

% of CD34/Vimentin expression measured by flow cytometry

TABLE 3
CAR-T Cell Activation

| | IFN-γ (pg/mL) from Treatment with Varying | |
|---|---|---|
| | Levels of CD34/Vimentin Expression | |
| CD34/Vimentin Expression | Control | CAR-T |
| None (0%) | 4 | 15 |
| Low (>0% to 25%) | 6 | 7345 |
| Mid (>25% to 75%) | 6 | 15678 |
| High (>75%) | 7 | 22165 |

% of CD34/Vimentin expression measured by flow cytometry

As can be seen in Tables 2 and 3, the higher the CD34/vimentin expression the greater the cell death, even low expression provided 65% cell death (by cell count), mid expression provided 81% cell death, and high expression provided 98% cell death.

It is to be understood that the above-referenced arrangements are illustrative of examples of the present disclosure. Thus, while the present technology has been described above in connection with the exemplary embodiments, it will be apparent to those of ordinary skill in the art that numerous modifications and alternative arrangements can be made without departing from the principles and concepts of the disclosure as set forth in the claims.

1. A method of treating cancer comprising disrupting activity of telocytes associated with the cancer.

2. The method of claim 1, wherein the telocytes express one or more of CD34, extracellular vimentin, c-kit, angiopoietin receptor, PDGFR-α and/or β, SCA-1, Podoplanin, caveolin-1, or telopods.

3. (canceled)

4. The method of claim 1, wherein the telocytes are capable of differentiating into cells expressing a smooth muscle actin.

5-11. (canceled)

12. The method of claim 1, wherein disrupting the activity of the telocytes is achieved through inhibition of ERK signaling.

13. The method of claim 12, wherein the inhibition of ERK signaling is achieved by induction of RNA interference against the ERK mRNA, administration of antisense oligonucleotides targeting ERK mRNA, administration of ribozymes targeting ERK mRNA, or induction of gene editing targeting the ERK gene.

14. The method of claim 13, wherein the RNA interference is induced by treatment with short interfering RNA or short hairpin RNA.

15-18. (canceled)

19. The method of claim 1, wherein inhibition of ERK signaling is achieved by administration of a small molecule ERK inhibitor.

20. The method of claim 19, wherein the ERK inhibitor is one or more of PD0325901, RDEA119, Olomoucine, Aminopurvalanol A, AS703026, AZD8330, BIX02188, BIX02189, CI-1040, Cobimetirlib, GDCs-0623, MEK162, PD318088, PD98059, Refametinib, R04987655,SCH772984, Selumetinib, SL327, Trametinib, ARRY-142886, or XL518.

21-41. (canceled)

42. The method of claim 1, wherein the telocytes express CAPN2, FHL2, and SOX1.

43. The method of claim 1, wherein the telocytes are inactivated and/or killed by generation of an immune response towards extracellular vimentin.

44. The method of claim 43, wherein the immune response is induced by a vaccination.

45. The method of claim 44, wherein the vaccination to induce the immune response towards extracellular vimentin is accomplished through administration of the vimentin protein in an immunogenic manner.

46. The method of claim 45, wherein the vimentin protein is administered through loading of dendritic cells in vitro and the dendritic cells are subsequently administered in vivo.

47. The method of claim 46, wherein the dendritic cells are generated from monocytes.

48. The method of claim 47, wherein the monocytes are plastic adherent.

49. The method of claim 47, wherein the monocytes express one or more of CD14, CD16, TLR4, TNF a upon stimulation, CD90, c-kit, c-met, CD25, PDGF-receptor, CD11b, or BDNF-receptor.

50-59. (canceled)

60. The method of claim 47, wherein the monocytes are treated with IL-4 and GM-CSF ex vivo to generate immature dendritic cells.

61. The method of claim 60, wherein the immature dendritic cells express:
one or more of IL-10 or CD11c;
lower levels of one or more of CD40, CD80, CD86, IL-12, IL-21, IL-18, IL-33, IL-15, or AIM2 as compared to mature dendritic cells;
higher levels of one or more of IL-35, TGF-β, HLA-G, ILT-3, ILT-4, LIF, inhibitor of kappa B, soluble TNF-α receptor, or interleukin-1 receptor antagonist as compared to mature dendritic cells; or
a combination thereof.

62-80. (canceled)

81. The method of claim 46, wherein the dendritic cells are induced to mature after administration in vivo.

82. The method of claim 81, wherein the maturation is induced by administration of one or more of Poly (IC), imiquimod, HMGB-1, CpG motifs, xenogeneic cell membranes, bacterial cell wall extract, p glucan, OK231, GM-CSF, neutrophil extracellular traps, free histones, yeast cell wall extract, KLH, zymosan, interferon γ, antibodies to IL-10 or its receptor, TNF-α, IL-33, β defensin, complement C3, complement C5, or necrotic cells.

83-103. (canceled)

104. The method of claim 1, wherein inactivation of the telocyte and/or induction of telocyte death is accomplished by vaccination via a vaccine including tumor telocytes.

105. The method of claim 104, wherein the tumor telocytes possess an increased ability to efflux rhodamine 123 as compared to telocytes isolated from non-malignant tissues.

106. The method of claim 104, wherein the vaccine is one or more of prophylactic, therapeutic, autologous, allogeneic, xenogeneic, generated from living telocytes, generated from mitotically inactivated telocytes, generated from telocyte necrotic particles, generated from telocytes that have undergone the process of pyroptosis, or generated from telocyte apoptotic bodies.

107-115. (canceled)

116. The method of claim 104, wherein the vaccine is generated from telocyte fusion with dendritic cells.

117. The method of claim 116, wherein the telocyte fusion with the dendritic cells is accomplished by electroporation, by treatment with polyethylene glycol, or using telocytes derived from pluripotent stem cells.

118-119. (canceled)

120. The method of claim 117, wherein the pluripotent stem cells are one or more of inducible pluripotent stem cells, embryonic stem cells, somatic nuclear transfer derived stem cells, or parthenogenic derived stem cells.

121-123. (canceled)

124. The method of claim 116, wherein the telocyte fusion with the dendritic cells is performed using telocytes derived from hematopoietic stem cells.

125. The method of claim 124, wherein the hematopoietic stem cells express one or more of CD34, CD133, Fas ligand, TRAIL receptor, AIM2, or notch.

126-130. (canceled)

131. The method of claim 116, wherein the telocyte fusion with the dendritic cells is performed using dendritic cells derived from pluripotent stem cells.

132. The method of claim 131, wherein the pluripotent stem cells include one or more of inducible pluripotent stem cells, embryonic stem cells, somatic nuclear transfer derived stem cells, or parthenogenic derived stem cells.

133-135. (canceled)

136. The method of claim 116, wherein the telocyte fusion with the dendritic cells is performed using dendritic derived from hematopoietic stem cells.

137. The method of claim 124, wherein the hematopoietic stem cells express one or more of CD34, CD133, Fas ligand, TRAIL receptor, AIM2, or notch.

138-142. (canceled)

143. The method of claim 116, wherein the fusion cell is activated prior to administration in a manner capable of increasing immunogenicity.

144. The method of claim 143, wherein the immunogenicity i-s includes an ability to evoke one or more of: a recall T cell immune response, a recall CD4 T cell immune response, a recall CD8 T cell immune response, a recall NK cell immune response, a recall NKT cell immune response, a recall γ delta cell immune response, or a recall neutrophil immune response, to one or more cancer telocyte induced antigens.

145-150. (canceled)

151. The method of claim 143, wherein the immunogenicity includes immunogenicity of a hybrid cell that is increased by exposure to one or more compounds that increase transporter associated protein expression.

152. The method of claim 151, wherein the one or more compounds include one or more of interferon α, interferon γ, TNF-α, interleukin-6, interleukin-12, or interleukin-18.

153-157. (canceled)

158. The method of claim 143, wherein the includes immunogenicity of a hybrid cell that is increased by exposure to one or more compounds that are increased by: MHC I and/or MHC II expression, transporter associated protein expression, exposure to one or more compounds capable of triggering the cGAS-STING pathway, exposure to one or more compounds capable of triggering the NOD pathway, or exposure to one or more compounds capable of triggering the toll-like receptor (TLR) pathway.

159. The method of claim 158, wherein the compound that increases transporter associated protein expression includes one or more of interferon α, interferon γ, TNF-α, interleukin-6, interleukin-12, or interleukin-18.

160-167. (canceled)

168. The method of claim 143, wherein the telocyte is grown in vitro and expanded before use as an immunogen or immunogenic source.

169. The method of claim 168, wherein the telocyte is isolated from one or more primary tumor sources.

170. The method of claim 169, wherein the tumor tissue of the one or more primary tomor sources is isolated and mechanically separated into smaller pieces than an original size of its tumor mass.

171. The method of claim 170, wherein the tumor tissue is separated and is mechanically dissected into multiple 1-3 mm pieces.

172. The method of claim 171, wherein the tumor tissue is treated with a tissue dissociating compound.

173. The method of claim 172, wherein the tumor dissociated compound i-s includes one or more of collagenase or trypsin/TrypLE.

174-175. (canceled)

176. The method of claim 173, wherein the tumor dissociated agent includes 0.05% collagenase and 0.1% trypsin/TrypLE.

177. The method of claim 172, wherein tissue is allowed to digest with incubation so as to allow the enzymes to digest the tissue.

178. The method of claim 177, wherein the tissue is exposed to the enzyme(s) for approximately 2-10 minutes.

179. The method of claim 178, wherein a mixture of the tissue and enzyme(s) is incubated in the presence of agitation.

180. The method of claim 179, wherein the agitation is performed by a shaker.

181. The method of claim 180, wherein an agitated solution of tissue and digestive enzymes is further dissociated by gentle pipetting.

182. The method of claim 181, wherein the gentle pipetting is performed in 1-30 minutes intervals.

183. The method of claim 182, wherein the supernatant is filtered through a series of meshes.

184. (canceled)

185. The method of claim 183, wherein the collected cell suspension is centrifuged at 10-150×g for 30 seconds to 5 minutes, the supernatant is then removed and re-centrifuged at 100-700×g for 20 seconds to 17 minutes, a pellet is obtained after centrifugation, the pellet is re-suspended in 1-15 mL of PEB medium to form a re-suspended pellet, a mixture of PEB and the re-suspended pellet is subsequently centrifuged at 20-45 g for 1-5 minutes, a supernatantant formed therefom is centrifuged at 100-300×g for 5-20 minutes to form a cell pellet.

186-188. (canceled)

189. The method of claim 185, the cell pellet is admixed with 0.01 to 10 mL of PEB and 1-5-μL of a c-kit antibody to form a cell pellet mixture, wherein the cell pellet mixture is incubated at 1-10° C. for 10-200 minutes and an additional additional 1-10 mL of PEB is added and then centrifuged at 100-700 g for 1-13 minutes.

190-192. (canceled)

193. The method of claim 189, wherein a pellet which results from centrifuging the cell pellet mixture is collected, re-suspended in 10-1000 μL of PEB and 1-200 μL of a solution containing goat anti-rabbit IgG microbeads, and incubated at 1-10° C. for 25-30 minutes to form an incubated mixture.

194-195. (canceled)

196. The method of claim 193, wherein the incubated mixture is added to a magnetic separation column in a magnetic field, and wherein unlabeled cells are allowed to pass through.

197. The method of claim 196, wherein the magnetic separation column is removed from the magnetic field, and wherein labeled cells are flushed out with PEB.

198. The method of claim 197, wherein the cells collected that disrupt activity of telocytes associated with cancer are washed and cultured in a media capable of allowing for growth of cancer specific telocytes.

199. The method of claim 198, wherein the media capable of allowing for growth of cancer specific telocytes includes one or more of DMEM containing 20% fetal calf serum, AIM-V media containing 20% fetal calf serum, RPMI media containing 20% fetal calf serum, EMEM media containing 20% fetal calf serum, or Iscove's media containing 20% fetal calf serum.

200-203. (canceled)

204. The method of claim 198, wherein the cancer specific telocytes are utilized as a cellular vaccine in an either therapeutic or prophylactic manner, wherein the cellular vaccine includes a telocyte population that has been gene transfected to augment immunogenicity.

205. The method of claim 204, wherein the telocyte population is mitotically inactivated.

206. The method of claim 205, wherein the telocyte population that is mitotically inactivated occurs as a result of one or more of exposure to ionizing radiation, a chemotherapeutic compound, or mitomycin-C.

207-208. (canceled)

209. The method of claim 204, wherein the telocyte population is gene transfected with one or more of interleukin-1 β, interleukin-1 β, interleukin-2, interleukin-4 and GM-CSF, GM-CSF, complement component c3, complement component c5, interleukin-7, interleukin-11, interleukin-12, interleukin-18, interleukin-21, interleukin-23, interleukin-27, interleukin-33, interferon α, interferon γ, interferon tau, interferon omega, TRAIL, BLyS, LIGHT, TNF-α, lymphotoxin, influenza surface antigen, PADRE epitope, HMGB1, short interfering RNA to interleukin-10, short interfering RNA to TGF-β, short interfering RNA to HLA-G, short interfering RNA to ILT-3, short interfering RNA to ILT-4, short interfering RNA to interleukin-1 receptor antagonist, short interfering RNA to interleukin-35, short interfering RNA to placental growth factor, short interfering RNA to VEGF, short interfering RNA to PDGF, short interfering RNA to sonic hedgehog, short interfering RNA to notch, or short interfering RNA to jagged.

210-247. (canceled)

248. The method of claim 204, wherein the telocyte population is gene edited to lack expression of interleukin-10.

249. The method of claim 204, wherein the telocyte population is gene edited to lack expression of one or more of TGF-β, HLA-G, ILT-3, ILT-4, interleukin-1 receptor antagonist, interleukin-35, placental growth factor, VEGF, PDGF, sonic hedgehog, notch, or jagged.

250-260. (canceled)

261. The method of claim 1, wherein the disrupting activity of the telocytes is achieved through creation of a lysate based cancer telocyte vaccine.

262. The method of claim 261, wherein the telocytes are generated from cancer tissues.

263. The method of claim 262, wherein the telocytes generated from the cancer tissues are expanded under conditions resembling the tumor microenvironment.

264. The method of claim 263, wherein the tumor microenvironment is replicated by culture of the tumor derived telocytes in hypoxia.

265. The method of claim 264, wherein the hypoxia is from 0.1%-10% by volume.

266. The method of claim 263, wherein the telocytes are expanded under conditions promoting acidosis.

267. The method of claim 266, wherein the conditions promoting acidosis are culture conditions possessing a pH of less than 7.2.

268. The method of claim 263, wherein the telocytes are cultured in the presence of one or more of TGF-β, PGE2, sialic acid, soluble TNF-α receptor p55, soluble TNF-α receptor p75, siglec-15, Galectin-1, Galectin-3, Galectin-7, Galectin-9, FGF-1, FGF-2, FGF-5, FGF-18, IGF-1, EGF-1, HGF, IL-17, IL-10, IL-4, IL-13, IL-35, VEGF-A, VEGF-C, PDGF-BB, angiopoietin, neutrophil extracellular traps, growth hormone, soluble HLA-G, carbon monoxide, radon gas, cobalt chloride, GITR ligand, RAE-1, embryonic stem cell conditioned media, GDF-5, GDF-11, thyroid stimulating hormone, oncostatin, plasminogen, or fibrinogen.

269-308. (canceled)

309. The method of claim 263, wherein the telocyte is utilized as a source of immunogen by extracting heat shock proteins and their bound immunogens from the telocyte.

310. The method of claim 263, wherein the telocyte and/or telocyte derived immunogens are administered into a patient with cancer in a manner to induce an immune response against cancer derived telocytes and/or immunogens derived from the telocytes, wherein the cancer patient is treated by the steps of:
a) identifying a patient suffering from cancer and possessing some degree of immune suppression;
b) administering a therapeutic capable of reducing tumor-associated immune suppression;
c) immunizing the patient with an immunogenic composition capable of expanding immune cells with tumor-targeting ability;
d) administering a localized a therapeutic capable of augmenting antigen presentation of cancer associated antigens from the cancer;
e) administering a therapeutic or treating the patient to induce localized tumor cell death; and
f) repeatedly administering an immunogenic composition capable of further expanding immune cells with tumor-targeting ability.

311. The method of claim 310, wherein the therapeutic capable of reducing tumor-associated immune suppression is an antioxidant from beta carotene, Vitamin E, anthocyanins, selenium, catechins, lutein, or lycopene.

312. The method of claim 311, wherein the antioxidant includes one or more of n-acetylcysteine, ascorbic acid, glutathione, vitamin k3, resveratrol, α lipoic acid, quercetin, kaempferol, myricetin, apigenin, luteolin, curcumin, caffeic acid.

313-324. (canceled)

325. The method of claim 310, wherein the therapeutic capable of reducing tumor-associated immune suppression is includes one or more of a phosphodiesterase (PDE-5) inhibitor, nitroglycerin, or a compound capable of reducing VEGF.

326. The method of claim 325, wherein the PDE-5 inhibitor is selected from Acetildenafi, Aildenafil, Avanafil, Benzamidenafil, Homosildenafil, Icariin, Lodenafil, Mirodenafil, Nitrosoprodenafil, Sildenafil, Sulfoaildenafil, Tadalafil, Udenafil, Vardenafil, Zaprinast, or a combination thereof.

327-328. (canceled)

329. The method of claim 325, wherein the compound capable of reducing the VEGF is selected from Avastin, Ciclopirox, penicillamine, tetrathiomolybdate, fish oil, selenium, green tea polyphenols, glycine, zinc, cirsimaritin, Eupafolin, Andrographolide, Procyanidin B2, Procyanidin B3, 6-O-angeloylenolin, Cyperenoic acid, Penduliflaworosin, Tylophorine, Ellagic acid, brucine, Punarnavine, Raddeanin A, Platycodin D, withanone, 4-Hydroxyphenylacetic acid, trans-ethyl p-methoxycinnamate, Decursin, decursinol angelate, Artesunate, or a combination thereof.

330. The method of claim 310, wherein the therapeutic capable of reducing tumor-associated immune suppression is a checkpoint inhibitor.

331. The method of claim 330, wherein the checkpoint inhibitor is a compound capable of blocking molecules selected from PD-1, PD-L1, CTLA-4, LAG-3, TIGIT, KIR, indolamine 2,3 deoxygenase, NR2F6, TIM-3, ILT-3, GITR, or a combination thereof.

332. The method of claim 310, wherein the patient is immunized with a tumor antigen, wherein the tumor antigen possesses similarity to the tumor which the patient is afflicted by.

333. The method of claim 332, wherein the tumor antigen is derived from a histologically similar tumor to which the patient is afflicted with.

334. The method of claim 333, wherein the tumor antigen is derived by one or more of lysis of histologically similar tumors, mRNA extraction of histologically similar tumors, or exosome extraction of histologically similar tumors.

335-336. (canceled)

337. The method of claim 332, wherein the tumor antigen is a tumor associated protein.

338. The method of claim 337, wherein the tumor associated protein is selected from Fos-related antigen 1, LCK, FAP, VEGFR2, NA17, PDGFR-β, PAP, MAD-CT-2, Tie-2, PSA, protamine 2, legumain, endosialin, prostate stem cell antigen, carbonic anhydrase IX, STn, Page4, proteinase 3, GM3 ganglioside, tyrosinase, MART1, gp100, SART3, RGS5, SSX2, Globol1, Tn, CEA, hCG, PRAME, XAGE-1, AKAP-4, TRP-2, B7H3, sperm fibrous sheath protein, CYP1B1, HMWMAA, sLe(a), MAGE A1, GD2, PSMA, mesothelin, fucosyl GM1, GD3, sperm protein 17, NY-ESO-1, PAX5, AFP, polysialic acid, EpCAM, MAGE-A3, mutant p53, ras, mutant ras, NY-BR1, PAX3, HER2/neu, OY-TES1, HPV E6 E7, PLAC1, hTERT, BORIS, ML-IAP, idiotype of b cell lymphoma or multiple myeloma, EphA2, EGFRvIII, cyclin B1, RhoC, androgen receptor, surviving, MYCN, wildtype p53, LMP2, ETV6-AML, MUC1, BCR-ABL, ALK, WT1, ERG (TMPRSS2 ETS fusion gene), sarcoma translocation breakpoint, STEAP, OFA/iLRP, Chondroitin sulfate proteoglycan 4 (CSPG4)329, or a combination thereof.

339. The method of claim 338, wherein a peptide or plurality of peptides derived from the antigens are used for immunization.

340. The method of claim 338, wherein a peptide or plurality of peptides used for immunization are matched with HLA haplotype of the patient in need of therapy.

341. The method of claim 338, wherein a peptide or plurality of peptides are altered peptide ligands.

342. The method of claim 338, wherein the immunization with the tumor antigen is performed together with an adjuvant.

343. The method of claim 342, wherein the adjuvant is a stimulator of antigen presentation.

344. The method of claim 343, wherein the stimulator of antigen presentation is a toll-like receptor (TLR).

345. The method of claim 344, wherein the toll-like receptor i-s includes one or more of TLR-2, TLR-3, TLR-4, TLR-5, TLR-7, TLR-8, or TLR-9.

346. The method of claim 345, wherein the TLR-2 is activated by compounds selected from, Pam3cys4, Heat Killed Listeria monocytogenes (HKLM), FSL-1, or a combination thereof.

347. (canceled)

348. The method of claim 345, wherein the TLR-3 is activated by Poly IC or double stranded RNA.

349. (canceled)

350. The method of claim 348, wherein the double stranded RNA is of prokaryotic origin.

351. The method of claim 350, wherein the double stranded RNA is derived from leukocyte extract.

352. The method of claim 351, wherein the leukocyte extract is a heterogeneous composition derived from freeze-thawing of leukocytes, followed by dialysis for compounds less than 15 kDa.

353. (canceled)

354. The method of claim 345, wherein the TLR-4 is activated by one or more of:
lipopolysaccharide,
peptide possessing at least 80 percent homology to the sequence:
EFDVILKAAGANKVAVIKAVRGATGLGLKEAKDLVESAPAALKEGVSKD DAEALKKALEEAGAEVEVK, HMGB-1, or
a peptide derived from HMGB-1.

355-357. (canceled)

358. The method of claim 354, wherein the HMGB-1 peptide is hp91.

359. (canceled)

360. The method of claim 345, wherein the TLR-5 is activated by flagellin.

361. (canceled)

362. The method of claim 345, wherein the TLR-7 is activated by imiquimod.

363. (canceled)

364. The method of claim 345, wherein the TLR-8 is activated by resmiqiumod.

365. (canceled)

366. The method of claim 345, wherein the TLR-9 is activated by CpG DNA.

367. The method of claim 310, wherein a stimulator of antigen presentation is added, wherein such a stimulator is a compound capable of upregulating expression of costimulatory molecules on antigen presenting cells.

368. The method of claim 367, wherein the costimulatory molecules are selected from CD40, CD80, CD86, or a combination thereof.

369. The method of claim 367, wherein the compound capable of upregulating expression of costimulatory molecules is an activator of NF-kappa B.

370. The method of claim 369, wherein the activator of NF-kappa B is an inhibitor of i-kappa B.

371. The method of claim 367, wherein the compound capable of inducing upregulation of costimulatory molecules is an activator of the JAK-STAT pathway.

372. The method of claim 369, wherein the activator of NF-kappa B is an activator of a Pathogen Associated Molecular Pattern (PAMP) receptor.

373. The method of claim 372, wherein the PAMP receptor is selected from MDA5, RIG-1, NOD, or a combination thereof.

374. The method of claim 310, wherein the compound capable of activating antigen presentation locally is a dendritic cell.

375. The method of claim 374, wherein the dendritic cell is:
activated with a TLR agonist or PAMP agonist,
generated from patient monocytes,
autologous to the patient in need of treatment.
allogeneic to the patient in need of treatment.

376-379. (canceled)

380. The method of claim 374, wherein the dendritic cell is activated in vivo by administration of one or more of GM-CSF or FLT-3L.

381. The method of claim 374, wherein the dendritic cell is activated in vivo by administration of FLT-3L.

382. The method of claim 310, wherein the means localized tumor cell death is achieved by one or more of administration of localized radiation therapy, cryoablation, localized administration of hyperthermia, or localized administration of chemotherapy.

383-385. (canceled)

386. The method of claim 382, wherein the chemotherapy includes administering compound selected from acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, fluorocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-n1, interferon alfa-n3, interferon β-I a, interferon γ-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride, or a combination thereof.

387. The method of claim 348, wherein the double stranded RNA is of mammalian origin.

388. The method of claim 1, wherein the telocyte is or includes FOXL1+, AQP1+, positive for an estrogen receptor, positive for a progesterone A receptor, LGR5+, or a combination thereof.

389-392. (canceled)

393. The method of claim 1, comprising administering a lysate-based cancer telocyte vaccine to a patient with cancer to induce an immune response against the cancer, wherein the lysate-based cancer telocyte vaccine includes telocytes from cancer tissue.

394. The method of claim 393, wherein the telocytes from the cancer tissue are expanded under conditions resembling the tumor microenvironment.

395. The method of 393, the patient is also afflicted with immune suppression.

396. The method of 395, further comprising administering a therapeutic capable of reducing tumor-associated immune suppression.

397. The method of claim 393, wherein administering the lysate-based cancer telocyte vaccine augments antigen presentation of cancer associated antigens from the cancer.

398. The method of claim 393, further comprising administering a therapeutic or treating the patient to induce localized tumor death.

399. The method of claim 393, wherein the lysate-based cancer telocyte vaccine includes a GM-CSF transfected telocyte.

400. A transfected telocyte lysate, wherein the transfected telocyte lysate is a lysate of telocytes transfected with one or more of interleukin-1β, interleukin-2, interleukin-4 and GM-CSF, GM-CSF, complement component c3, complement component c5, interleukin-7, interleukin-11, interleukin-12, interleukin-18, interleukin-21, interleukin-23, interleukin-27, interleukin-33, interferon α, interferon γ, interferon tau, interferon omega, TRAIL, BLyS, LIGHT, TNF-α, lymphotoxin, influenza surface antigen, PADRE epitope, HMGB1, short interfering RNA to interleukin-10, short interfering RNA to TGF-β, short interfering RNA to HLA-G, short interfering RNA to TLT-3, short interfering RNA to TLT-4, short interfering RNA to interleukin-1 receptor antagonize, short interfering RNA to interleukin-35, short interfering RNA to placental growth factor, short interfering RNA to VEGF, short interfering RNA to PDGF, short interfering RNA to sonic hedgehog, short interfering RNA to notch, and/or short interfering RNA to jagged, for example. In other examples, the telocyte population can be gene edited to lack express of interleukin-10, TGF-β, HLA-G, ILT-3, ILT-4, interleukin-1 receptor antagonist, interleukin-35, placental growth factor, VEGF, PDGF, sonic hedgehog, notch, or jagged.

401. The transfected telocyte lysate of claim 400, wherein the telocytes are formulated in a cancer therapeutic vaccine, are transfected with at least GM-CSF, or a combination thereof.

402-403. (canceled)

404. A hybrid cell composition, comprising:
a leukocyte-containing cellular culture including monocytes, dendritic cells, effector T cells, or a combination thereof; and
a telocyte lysate fused with cells or cellular material of the leukocyte-containing cellular culture.

405. The hybrid cell composition of claim 404, wherein the hybrid cell composition expresses CD34 along with vimentin, vascular endothelial growth factor (VEGF), or both.

406. (canceled)

407. The hybrid cell composition of claim 404, wherein the leukocyte-containing cellular culture includes one or more of at least 50% dendritic cells by cell count, at least 50% monocytes by cell count, or at least 50% effector T cells by cell count.

408-409. (canceled)

410. The hybrid cell composition of claim 404, wherein dendritic cells are present and are immature dendritic cells.

411. The hybrid cell composition of claim 404, wherein the telocyte lysate includes telocytes from cancer tissue.

412. The hybrid cell composition of claim 404, further comprising a therapeutic compound capable of reducing tumor-associated immune suppression, inducing localized tumor death, or both.

413. (canceled)

414. The hybrid cell composition of claim 404, in the form of a vaccine.

415. The hybrid cell composition of claim 414, wherein the telocyte lysate of the vaccine is,
generated from living telocytes, mitotically inactivated telocytes, telocyte necrotic particles, telocytes after pyroptosis, telocyte apoptotic bodies, telocytes derived from pluripotent stem cells,
electrically pulse-fused with the cells or cellular material of the leukocyte-containing cellular culture,
ultrasonically pulse-fused with the cells or cellular material of the leukocyte-containing cellular culture, or
fused with the cells or cellular material of the leukocyte-containing cellular culture is by treatment with polyethylene glycol, or
generated from telocytes grown in vitro and expanded as an immunogen or immunogenic source, under conditions promoting acidosis, under conditions resembling the tumor microenvironment, or a combination thereof.

416-419. (canceled)

420. The hybrid cell composition of claim 404, further comprising a cancer growth media capable of allowing for growth of cancer specific telocytes, wherein the cancer growth media includes DMEM media, AIM-V media, RPMI media, EMEM media, Iscove's media, or a combination thereof.

421. The hybrid cell composition of claim 404, wherein the telocytes are cultured in the presence of one or more of TGF-β, PGE2, sialic acid, soluble TNF-α receptor p55, soluble TNF-α receptor p75, siglec-15, Galectin-1, Galectin-3, Galectin-7, Galectin-9, FGF-1, FGF-2, FGF-5, FGF-18, IGF-1, EGF-1, HGF, IL-17, IL-10, IL-4, IL-13, IL-35, VEGF-A, VEGF-C, PDGF-BB, angiopoietin, neutrophil extracellular traps, growth hormone, soluble HLA-G, carbon monoxide, radon gas, cobalt chloride, GITR ligand, RAE-1, embryonic stem cell conditioned media, GDF-5, GDF-11, thyroid stimulating hormone, oncostatin, plasminogen, or fibrinogen.

422. The hybrid cell composition of claim 404, further comprising a stimulator of antigen presentation capable of upregulating expression of costimulatory molecules on antigen presenting cells.

423. A modified T cell, wherein the modified T cells includes chimeric multi-antigen receptors.

424. The modified T cell of claim 423, wherein the chimeric multi-antigen receptors are at least bi-specific for one or more of CD34/Vimentin, CD34/VEGF, CD34/PDTF.

425-426. (canceled)
